(19) United States
(12) Patent Application Publication
(10) Pub. No.: US 2025/0378953 A1
(43) Pub. Date: Dec. 11, 2025

(54) ORGAN DIGITAL TWIN SYSTEMS AND METHODS FOR CREATING AND USING SUCH SYSTEMS

(71) Applicant: Yale University, New Haven, CT (US)

(72) Inventors: Gregory Tietjen, New Haven, CT (US); Jenna DiRito, New Haven, CT (US); Owen Richfield, New Haven, CT (US); Alborz Feizi, New Haven, CT (US); Daniel Jordan Viray, New Haven, CT (US); Thomas Hefele, New Haven, CT (US)

(21) Appl. No.: 18/877,978

(22) PCT Filed: Jun. 21, 2023
(86) PCT No.: PCT/US2023/068833

§ 371 Date: Dec. 20, 2024

Related U.S. Application Data
(60) Provisional application No. 63/354,012, filed on Jun. 21, 2022.

(51) Int. Cl. G16H 50/20; A01N 1/143

(52) U.S. Cl. CPC G16H 50/20; A01N 1/143

(57) ABSTRACT

Improved apparatuses, systems, and/or methods for collecting and using human organ data are disclosed. The apparatuses, systems, and/or methods involve (a) collecting one or more classes of biological data from an ex vivo or in vivo (e.g. in situ) normothermic perfused instance of an isolated organ of interest; and (b) storing the collected biological data in an organ digital twin of the organ of interest, to logically connect modes of organ failure to means of effective therapeutic intervention. The apparatuses, systems, and/or methods can also incorporate a machine learning module to compute the current health state for the instance of the organ of interest.

### CROSS-REFERENCE TO RELATED APPLICATIONS

This application claims benefit of and priority to U.S. Ser. No. 63/354,012 filed Jun. 21, 2022, which is incorporated herein by reference in its entirety.

### STATEMENT REGARDING FEDERALLY SPONSORED RESEARCH

This invention was made with government support under DK124420 and DK128662 awarded by National Institutes of Health. The government has certain rights in the invention.

### FIELD OF THE INVENTION

This invention is generally related to organ perfusion, particularly continuous ex vivo normothermic machine perfusion of human organs and use of digital data from the process for real-time decision-making in clinical settings involving organ transplant and in research settings as a drug development platform.

### BACKGROUND OF THE INVENTION

Chronic organ disease and/or organ failure represent a massive and growing health and economic burden in the U.S., accounting for >$400 billion in current health care spending per year. The only cure is organ transplantation. Currently, the demand for implantable human organs (such as kidneys, livers, hearts, and lungs, among others) exceeds the available supply, resulting in thousands of patient deaths per year in the United States alone. One reason for the shortage is that donated organs are often discarded, because they are deemed unsuitable for transplant due to low viability scores, the underlying health of the donor, and in some cases, measurement or systemic errors. Another reason is the lack of alternative therapeutic options to organ transplantation resulting from the extremely slow and ineffective process of developing novel therapies for organ-centered diseases. Further, even non-transplantable organs have valuable information to probe for the purposes of developing therapeutic alternatives to organ transplantation. As a result, the opportunity to use every potentially viable donated organ either for a life-saving transplant or to accelerate and de-risk the process of drug development to provide alternatives to organ transplant is not being realized. A key bottleneck towards realizing this potential is the absence of data collected on a large number of specific types of organs (e.g., human organs) in isolation from the background noise of other organs in a patient; and systems to collect, process, store these data, and/or provide the data in real-time for clinical decision-making. Similarly, these data have not been leveraged to develop pipelines for new drugs to maintain and/or treat organs ex vivo and/or in vivo.

Regarding real-time decision-making in clinical settings of organ transplant, the process is currently hampered by the outmoded analog (such as pen and paper) way ex vivo organ data are collected. Further, it is cumbersome to share these data, and the only way to solicit expert help from a device company by sharing of images of these analog data. Even when such data are shared, they are limited to a small sample size per organ. This means that accurate and fast diagnosis of modes of organ failure and/or organ diseases remain challenging and longer times could be spent to confirm diagnosis and/or identify improved and/or optimal therapies to render an organ safe for transplantation.

Regarding drug discovery and development, on average, it costs $1 billion and approximately 12-14 years to bring a new drug to market. Moreover, ˜90% of drugs that make it to phase 1 clinical trials fail. Further, costs and prior negative efficacy data are often insurmountable barriers for repurposing a “failed” drug for a different indication. In addition, drugs that do get approved can cause as many issues as the cure, particularly with serious diseases like chronic kidney disease. Thus, a major bottleneck in the drug development pipeline exists leading to major challenges in developing alternatives to organ transplantation. An over reliance on mouse and cell culture models of diseases has created this critical bottleneck. It is possible to engineer molecular specificity in a variety of drug classes (small molecules, biologics, RNA). But getting these molecules to the specific site of need, at the right time, in the right duration, for the right patient presents several challenges. The lack of logistical control over drug delivery leads to a wide array of serious side effects. While mouse and cell culture can replicate the molecular basis of some human disease, they fail to recapitulate natural human variability, anatomy, and physiology, and cannot replicate the complex nature of human organ failure. Another problem is that the use of the typical genetically identical animals leads to advancement of drugs with low efficacies that, while apparent in homogenous animal models, fail to show efficacy in the heterogeneous human population—which is another reason that clinical trials fail.

Accordingly, there remains a major unmet need to develop platforms (i) for improved real-time decision-making in clinical settings involving organ transplant and/or (ii) drug discovery/development programs that capture the variability, anatomy, and physiology of human organs.

Therefore, it is an object of the invention to provide improved apparatuses, systems, and/or methods in organ transplant settings, such as in clinical organ transplant settings.

It is also an object of the invention to provide new platforms for drug discovery and development, which recapitulate human variability, anatomy and physiology.

### SUMMARY OF THE INVENTION

Disclosed are improved apparatuses, systems, and/or methods for collecting and using human organ data. The apparatuses, systems, and/or methods involve (a) collecting one or more classes of biological data from an ex vivo or in vivo (e.g. in situ) normothermic perfused instance of an isolated organ of interest; and (b) storing the collected biological data in an organ digital twin of the physical organ of interest. The purpose of the organ digital twin is to logically connect modes of organ failure to means of effective therapeutic intervention. Several organs are amendable to the disclosed apparatuses, systems, and/or methods. These include, but are not limited to, lungs, kidneys, livers, and hearts. In some forms, the apparatuses, systems, and/or methods involving transmitting all or a portion of the data contained in the organ digital twin and/or all or a portion of data derived from the organ digital twin, and using a machine learning module to compute the current health state for the instance of the organ of interest.

Disclosed are methods that include (a) collecting one or more classes of biological data from an ex vivo or in vivo (e.g. in situ) normothermic perfused instance of an isolated organ of interest, and (b) storing the collected biological data in an organ digital twin of the organ of interest. In some forms, the methods further include storing donor data in the organ digital twin, wherein the donor data is data related to the donor of the instance of the organ of interest. In some forms, the methods further include storing point of care data in the organ digital twin, wherein the point of care data is data collected from the instance of the organ of interest before, during, or after the period of the ex vivo or in vivo (e.g. in situ) perfusion of the instance of the isolated organ of interest. Also disclosed are platforms that incorporate one or more of these methods. These platforms involve collecting biological data from an ex vivo or in vivo (e.g. in situ) normothermic perfused isolated organ, and contain an organ perfusion machine, and an edge device communicatively coupled to the organ perfusion device. Accordingly, also described is an edge device configured for communication between one or more medical-grade perfusion devices, one or more pieces of diagnostic equipment used in combination with the perfusion devices, and/or one or more data storage platforms. The edge device captures biological data from the perfusion machine during perfusion of the ex vivo or in vivo (e.g. in situ) normothermic perfused isolated organ as well as other types of point of care or real-time diagnostic tools. Preferably, the edge device contains custom software to interface with the organ perfusion machine and diagnostic equipment, collect data from the organ perfusion machine and diagnostic equipment, and upload the data to one or more data storage platforms and/or graphical user interfaces for visualization. The data can be collected real time for interactive visualization and/or creation of an organ digital twin.

In some forms, the classes of biological data include one or more of physiologic data, genomic data, transcriptomic data, metabolomic data, proteomic data, lipidomic data, biopsy data, histological data, physical condition data, organ perfusion data, organ management data, and organ treatment data.

In some forms, the physiologic data includes one or more of arterial pressure, arterial flow, venous pressure, venous flow, organ-specific functional assessment (e.g., urine production/quality in a kidney, bile production/quality in a liver), and blood gas analysis. In some forms, the blood gas analysis includes one or more of pH, pO2, pCO2, Base Excess, bicarbonate, total carbonate, lactate, hematocrit, hemoglobin, BUN, potassium, sodium, chloride, and anion gap.

In some forms, the genomic data includes one or more of whole exome sequencing and whole genome sequencing. In some forms, the transcriptomic data includes one or more of bulk RNA sequencing, single cell RNA sequencing, single nuclear RNA sequencing, spatial RNA sequencing, and fluorescent in situ hybridization. In some forms, the metabolomic data includes one or more of unbiased metabolomics, targeted analysis, metabolite profiling, metabolic fingerprinting, and spatial metabolomic imaging. In some forms, the proteomic data includes one or more of targeted protein microarrays, ELISA, unbiased proteomics, Luminex assays. In some forms, the lipidomic data includes one or more of direct infusion mass spectrometry (MS) analysis (also known as shotgun lipidomics), liquid-phase separations coupled to MS (typically liquid chromatography (LC-MS)), and desorption ionization techniques MS approaches (often used for mass spectrometry imaging (MSI)).

In some forms, the histological data includes data generated using one or more of formalin fixed samples, paraffin embedded samples, fresh tissue section, frozen tissue section, histologic staining, histological imaging, standard histochemical stain, immunohistochemical stain, immunofluorescent stain, confocal microscopy, two-photon microscopy, epifluorescence, and light sheet microscopy. In some forms, the standard histochemical stain is H&E, PAS, MSB, or Trichrome. In some forms, the immunohistochemical stain is against one or more proteins of interest and/or against one or more cellular processes of interest.

In some forms, the physical condition data includes one or more of anatomic information related to the donor or anatomic information related to the instance of the organ, damage to the instance of the organ associated with recovery and preservation of the organ prior to initiation of perfusion, damage to the instance of the organ during perfusion, and damage to the instance of the organ following perfusion.

In some forms, the organ perfusion data includes one or more of arterial pressure, arterial flow, venous pressure, venous flow, organ-specific functional assessment (e.g. urine production/quality in a kidney, bile production/quality in a liver), and blood gas analysis. In some forms, the blood gas analysis includes one or more of pH, pO2, pCO2, base excess, O2 saturation, bicarbonate, total carbon dioxide, lactate, hematocrit, hemoglobin, BUN, potassium, sodium, chloride, ionized calcium, and anion gap.

In some forms, the organ management data includes one or more of volume addition, mix of blood cells, crystalloid, and colloid in volume addition, volume removal, dialysis flow rate in, dialysis flow rate out, composition of dialysate, surgical intervention, nutritional maintenance, and additional maintenance infusion. In some forms, the surgical intervention includes one or more of cautery and sutures. In some forms, the nutritional maintenance includes one or more of type of nutrition and flow rate of infusion. In some forms, the additional maintenance infusion includes one or more of bile salts and heparin.

In some forms, the organ treatment data includes one or more of interventions to be evaluated. In some forms, the intervention includes one or more perturbations of the system including administration of drugs such as small molecules, nucleic acids, biologics and/or nanomedicines of various compositions. In some forms, the intervention is chosen to establish or distinguish the link between modes of failure and types of intervention.

In some forms, the donor data includes one of more of blood gas analysis prior to organ recovery, labs prior to organ recovery, and summary data of the donor demographics and history.

In some forms, the point of care data includes one of more of blood gas analysis, metabolic panel analysis, and GEM blood analyzer. In some forms, the blood gas analysis is collected from one or more of iSTAT, CHEM8, and CG4+. In some forms, the metabolic panel analysis is from PICOLLO system. In some forms, the blood analysis includes one or more of freezing point osmometer and biomarker analysis.

In some forms, prior to storing the data, the donor data, point of care data, and/or one or more classes of biological data included in the organ digital twin, is collected from or obtained for one or more different instances of the organ of interest, wherein the organ digital twin comprising data of the instance of the organ of interest and data collected from or obtained for one or more different instances of the organ of interest constitutes a collective organ digital twin.

In some forms, the donor data, point of care data, and biological data stored in the organ digital twin is collected from or obtained for only the instance of the organ of interest, wherein the organ digital twin comprising data of the instance of the organ of interest constitutes an individual organ digital twin.

In some forms, the methods further include analyzing the data in the organ digital twin to determine the condition of the instance of the organ of interest. In some forms, an alert is generated if the condition of the instance of the organ of interest determined by the analysis of the organ digital twin indicates that the instance of the organ of interest needs mechanical or therapeutic intervention. In some forms, the methods further include altering the ex vivo or in vivo (e.g. in situ) perfusion conditions for the instance of the isolated organ of interest based on the condition of the instance of the organ of interest determined by the analysis of the organ digital twin. In some forms, the methods further include performing a mechanical intervention on the instance of the organ of interest based on the condition of the instance of the organ of interest determined by the analysis of the organ digital twin. In some forms, the methods further include treating the instance of the organ of interest based on the condition of the instance of the organ of interest determined by the analysis of the organ digital twin to rehabilitate the instance of the organ of interest.

In some forms, the methods further include analyzing the data in the organ digital twin to determine one or more modes of failure of the instance of the organ of interest. In some forms, the methods further include treating, ex vivo and/or in vivo (e.g., in situ), the instance of the organ of interest based on one or more of the determined modes of failure.

In some forms, analyzing the data identifies a logical connection between one or more modes of organ failure to one or more forms of therapeutic intervention. In some forms, the determined modes of failure are in one or more classes of modes of failure. In some forms, the classes of modes of failure include one or more of perfusion device failure modes, vascular failure modes, metabolic failure modes, immunological failure modes, and surgical failure modes. In some forms, the perfusion device failure modes include one or more of hypotension, hemorrhage, low hematocrit, low pH, and high potassium. In some forms, the vascular failure modes include one or more of venous hypertension, arterial hypertension, non-device-related hypotension, edema, and microvascular obstruction. In some forms, the metabolic failure modes include one or more of lactic acidosis, metabolic alkalosis, respiratory acidosis, respiratory alkalosis, and succinate-mediated electron transport disruption. In some forms, the immunological failure modes include one or more of dysfunctional regulated cell death, dysfunctional IL-1-mediated inflammation, dysfunctional TNF-mediated inflammation, and excessive damage-associated molecular pattern release. In some forms, the excessive damage associated molecular pattern release involves release of one or more of HMGB1, cell free DNA, ATP, and uric acid.

In some forms, the methods further include treating the instance of the organ of interest with a proposed therapy, collecting additional biological data from the instance of the organ of interest, storing the collected additional biological data in the organ digital twin, and analyzing the data in the organ digital twin to determine one or more of the effects of the proposed therapy on the instance of the organ of interest. In some forms, the methods further include repeating the treating, collecting, storing, analyzing, and determining steps with a second proposed therapy. In some forms, the methods further include storing the determined effects in the organ digital twin.

In some forms, the methods further include analyzing the data in the organ digital twin to predict one or more effects on the organ of interest of a therapy of interest. In some forms, the methods further include treating the instance of the organ of interest with the therapy of interest, collecting additional biological data from the instance of the organ of interest, storing the collected additional biological data in the organ digital twin, and analyzing the data in the organ digital twin to determine one or more of the effects of the therapy of interest on the instance of the organ of interest. In some forms, the methods further include storing the determined effects of the therapy of interest in the organ digital twin.

In some forms, the methods further include analyzing the data in the organ digital twin to predict one or more effects on the organ of interest of a therapy of interest. In some forms, the methods further include treating the instance of the organ of interest with the therapy of interest, collecting additional biological data from the instance of the organ of interest, storing the collected additional biological data in the organ digital twin, and analyzing the data in the organ digital twin to determine one or more of the effects of the therapy of interest on the instance of the organ of interest. In some forms, the methods further include repeating the treating, collecting, storing, analyzing, and determining steps with a second therapy of interest. In some forms, the methods further include storing the determined effects of the therapy of interest in the organ digital twin.

In some forms, the methods further include integrating transplant recipient data with all or a portion of the data comprised in the organ digital twin and/or all or a portion of data derived from the organ digital twin. In some forms, the methods further include analyzing the integrated transplant recipient data and organ digital twin data to assess suitability of the instance of the organ of interest for transplant into the recipient.

In some forms, the methods further include transmitting and/or displaying, in real time and/or as a static record, all or a portion of the data comprised in the organ digital twin and/or all or a portion of data derived from the organ digital twin. In some forms, the data is transmitted, wirelessly or via a wired network, to one or more remote devices. In some forms, the displaying comprises displaying the transmitted data on the remote device. In some forms, the data derived from the organ digital twin comprises the condition of the instance of the organ of interest, a generated alert, altered ex vivo or in vivo (e.g. in situ) perfusion conditions, a mechanical intervention, and/or actions or interventions suggested by the analysis of data in the organ digital twin.

In some forms, the methods further include analyzing all or a portion of the data comprised in the organ digital twin to produce derivatized data from the organ digital twin.

Also disclosed are methods of modeling responses of the organ of interest involving analyzing the response of any of the disclosed organ digital twins to an action of interest. In some forms, the action of interest is a change in one or more of the data comprised in the organ digital twin and/or in one or more of the data derived from the organ digital twin.

In some forms, the organ digital twin is stored in a digital physical medium. In some forms, the data is stored by writing the data in a digital physical medium. In some forms, the storage is performed by a computer configured to accomplish the storage.

In some forms, the methods further include transmitting all or a portion of the data comprised in the organ digital twin and/or all or a portion of data derived from the organ digital twin, and using a machine learning module to compute the current health state for the instance of the organ of interest. In some forms, the machine learning module is configured to train a machine learned model based on the transmitted data. In some forms, the transmitted data includes donor data, point of care data, and/or one or more classes of biological data. In some forms, computing the current health state for the organ of interest comprises using enriched data. In some forms, the enriched data is from the same type of organ from the same donor or different donors. In some forms, the machine learning module is configured to train a machine learned neural network model, a Bayesian model, an artificial intelligence system, a rules-based system, or a combination thereof. In some forms, the machine learning module trains the models in a supervise, unsupervised, or semi-supervised manner. In some forms, the machine learning module comprises neural networks selected from recurrent neural networks, convolutional neural networks, and artificial neural networks.

Also disclosed are organ digital twins produced by any of the disclosed methods.

### BRIEF DESCRIPTION OF THE DRAWINGS

FIG. 1 is a flow diagram illustrating a non-limiting organ digital twin ecosystem.

FIG. 2 is an image of a non-limiting illustration of a digital interface on an exemplary perfusion machine.

FIG. 3 is an image of an exemplary manual data sheet.

FIG. 4 is an image of a non-limiting illustration of an isolated organ.

FIG. 5 is an image of a non-limiting illustration of an organ digital twin.

FIGS. 6-9 are images of non-limiting illustrations of perfusion machines.

FIG. 10 is a flow diagram of a non-limiting method for creating an organ digital twin.

FIG. 11 is a collection of line graphs showing interactive data visualization developed for real-time monitoring and analysis of organ perfusion using porcine kidney. Values not displaying are organ donor information, which are unavailable for the porcine kidney perfusion.

FIG. 12 is a collection of line graphs showing continuous monitoring of a kidney following histamine administration.

### DETAILED DESCRIPTION OF THE INVENTION

### I. Definitions

“Digital twin” or “organ digital twin” may refer to data containing the biological data obtained from an isolated human organ stored on an electronic device, or to a computer model created to approximate and characterize the operation, response to stress and/or therapy, and/or health status of an isolated human organ based on measurement data, perfusion data, demographic data, anatomic, and/or other data or information specific to that particular isolated organ.

“Isolated organ” may refer to a human organ in a biologically active state during preservation, storage, and/or perfusion, including organs that are in an ex vivo and/or in vivo (e.g. in situ) setting within a human body (living or deceased).

### II. Organ Digital Twins

To address the problems discussed above, improved apparatuses, systems, and/or methods for collecting and using organ data are disclosed. In general, the apparatuses, systems, and/or methods involve (a) collecting one or more classes of biological data from an ex vivo or in vivo (e.g. in situ) normothermic perfused instance of an isolated organ of interest; and (b) storing the collected biological data in an organ digital twin of the organ of interest. In some forms, the biological data is deidentified. Also described is an organ digital twin produced by any of the methods described herein. Preferably, in some forms, the purpose of the organ digital twin is to logically connect modes of organ failure to means of effective mechanical and/or therapeutic intervention, preferably to means of effective therapeutic intervention.

Ex vivo or in vivo (e.g. in situ) perfusion of human organs combined with organ digital twin technology can be used to achieve both clinical translation as well as drug development. Transplant-declined human organs may be perfused at normothermic temperatures outside the body for at least five days (for example, periods as long as 7-10 days or more), thereby providing an ideal platform for discovering, testing, and translating of new drugs, particularly those intended to treat acute or chronic organ disease and organ failure. The disclosed systems, methodologies, and apparatuses involve ex vivo or in vivo (e.g. in situ) perfusion of isolated human organs in connection with a fully automated and real-time system for comprehensive data capture, integration and interpretation to create a digital replica of every aspect of organ physiologic function (for example, blood flow, pressure, blood gas analysis, etc.) perturbations (for example response to injury, disease, etc.) and response to therapy.

### i. Immortalization of Organ Data

In some forms, the apparatuses, systems, and/or methods involve storing donor data in an organ digital twin of the organ. The donor data is related to the donor of the instance of the organ of interest, is related to the donor of the instance of the organ of interest, or a combination thereof. In some forms, the donor data is related to the donor of the instance of the organ of interest. In some forms, the donor data is deidentified.

In some forms, the apparatuses, systems, and methods for facilitate organ the generation of a digital twin (such as computer models), such that representations of individual organs may be utilized for predictions, drug development testing, organ harvesting, or organ revitalization in a clinical setting. In some forms, the apparatuses, systems, and/or methods include perfusing transplant-declined organs (for example, lungs, kidneys, livers, and hearts, among others) on a perfusion machine, capturing data from the machine during organ perfusion, uploading the data via a device (such as an edge device) to a transplant-declined human organ database (THOD), building a computer model (such as a digital twin) of the individual organ based on the uploaded data, and using the digital twin for useful follow-on actions such as treatment predictions, drug development testing, and organ revitalization. In some forms, data captured during perfusion and subsequent analysis thereof may result in the organ being deemed viable for transplant.

In some forms, the apparatuses, systems, and/or methods involve creating an organ digit twin, the methods involving: installing an isolated organ within a perfusion machine; communicatively coupling the perfusion machine to a device (such as an edge device); communicatively coupling the device (such as an edge device) to a digital organ database; capturing functional data from the isolated organ during perfusion, the functional data being transmitted to the digital organ database via the device (such as an edge device); assessing at least one failure mode of the isolated organ; and immortalizing the organ digital twin upon completion of perfusion of the isolated organ.

In some forms, the apparatuses, systems, and/or methods involve storing point of care data in the organ digital twin. The point of care data can be data collected from the instance of the organ of interest before, during, or after the period of the ex vivo or in vivo (e.g. in situ) perfusion of the instance of the organ of interest.

In some forms, the classes of biological data include one or more of physiologic data, genomic data, transcriptomic data, metabolomic data, proteomic data, lipidomic data, biopsy data, histological data, physical condition data, organ perfusion data, organ management data, organ treatment data, quantitative microscopy data, whole organ imaging data, partial organ imaging data, bulk DNA sequencing data, bulk RNA sequencing data, and single cell analyses data. Preferably, these data are captured on an isolated organ.

The categories, classes, and types of data that can be stored, analyzed, immortalized, etc. in and in relation to the disclosed organ digital twins, systems, methods, etc. are organized and labelled herein solely for convenience and to simplify discussion of the forms of data that can be used with the disclosed technology. The majority of categories and classes of data described herein relate to traditional categorizations of data, typically based on the type of date (for example, physiological data relates to physiology, donor data relates to the donor). However, other organizations are possible and are contemplated herein. For example, categories of data can be organized by the source of the data. For example, one such source-and stage-based categorization is donor derived data, organ data derived prior to perfusion, organ data derived during perfusion, tissue biopsy derived data, perfusate derived data, and bodily fluid derived data. Importantly, similar data, such as blood gas analysis data can be collected or derived form multiple different sources and at multiple different stages. Every type of data that can be assessed, collected, or derived from any source and/or during any stage in the described organ digital twins, systems, methods, devices, components, materials, and data sets is specifically contemplated as data that can be collected, derived, analyzed, stored, immortalized, transmitted, displayed, etc., in appropriate forms, as part of the disclosed organ digital twins, systems, methods, devices, components, materials, and data sets.

Physiologic data includes, but is not limited to, arterial pressure/flow, arterial pressure, arterial flow, venous pressure, venous flow, organ-specific functional assessment (e.g. urine production/quality in a kidney, bile production/quality in a liver), and blood gas analysis including, but not limited to—pH, partial oxygen pressure (pO₂), partial carbon dioxide pressure (pCO₂), base excess, O2 saturation, bicarbonate, total carbon dioxide, lactate, hematocrit, hemoglobin, blood urea nitrogen (BUN), potassium, sodium, chloride, ionized calcium, and anion gap).

Genomic data includes, but is not limited to, whole exome sequencing, whole genome sequencing, etc.

Transcriptomic data includes, but is not limited to, bulk RNA sequencing, single cell RNA sequencing, single nuclear RNA sequencing, spatial RNA sequencing, fluorescent in situ hybridization, etc.

Metabolic data includes, but is not limited to, unbiased metabolomics, targeted analysis, metabolite profiling and metabolic fingerprinting on bulk or single cell samples, spatial metabolomic imaging, etc.

Biopsy data generally refers to data that is obtained from a physically excised subset of a tissue. Thus, it can include the proteomic, metabolomic, glycomic, genomics, transcriptomic, etc., data described herein. The data can be obtained from the donor; organ prior to perfusion; organ during perfusion; organ after perfusion; tissue biopsy; perfusate; bodily fluid, or a combination thereof.

Histological data includes, but is not limited to, data obtained from formalin fixed and paraffin embedded samples for histologic staining and imaging involving, but not limited to, standard histochemical stains such as hematoxylin and eosin (H&E) stain, periodic acid-Schiff (PAS) stain, Martius Scarlet Blue (MSB) stain, Trichrome, etc.; immunohistochemical stains against proteins of interest (e.g., fibrinogen, caspase 3, etc.) and/or cellular processes of interest (e.g., TUNEL stain). Histological data can also include immunofluorescent data captured on fresh or frozen tissue sections. This can include, confocal microscopy, two-photon microscopy, epifluorescence, light sheet microscopy etc.

Physical condition data includes, but is not limited to, structural and appearance condition of the organ, such as anatomic information unique to donor organ, the donor, and any damage associated with recovery and preservation of the donor organ prior to, during, or after initiation of normothermic machine perfusion.

Organ management data is intended to include data related to the perfusion machine settings and other data related to perfusion that is not about the organ itself (e.g., flow rate). Organ management data also includes organ perfusion data, which involves data about the organ's perfusion characteristics, such as arterial pressure/flow, arterial pressure, arterial flow, venous pressure, venous flow, and blood gas analysis including, but not limited to—pH, pO₂, partial carbon dioxide pressure pCO₂, base excess, O2 saturation, bicarbonate, total carbon dioxide, lactate, hematocrit, hemoglobin, BUN, potassium, sodium, chloride, ionized calcium, and anion gap. Further the organ management data includes the data associated with what is done to the organ in real time to keep the organ perfusion data in a normal physiologic range. These can include, but are not limited to, volume addition (with breakdown of mix of blood cells, crystalloid, colloid), volume removal, dialysis with in/out flow rates and composition of dialysate, surgical intervention (e.g., cautery, sutures), nutritional maintenance (type of nutrition, flow rate of infusion), additional maintenance infusions (e.g., bile salts, heparin).

Organ treatment data includes, but is not limited to, drugs, fluids, reagents, and physical perfusion regimes other than those for normal management of the organ. Treatment regimens would be associated with particular therapeutic interventions being evaluated. The purposes of these therapeutic interventions can be described as perturbations of the system to uncover the link between modes of failure and means of intervention.

Organ donor data includes, but is not limited to, all the data available about an organ donor in the standard context of organ donation. This includes all blood gas analysis and labs during the hospital stay prior to organ recovery and summary data of the donor demographics and history. Preferably, the organ donor data is de-identified.

Point of care data includes, but is not limited to, blood gas analysis, such as that collected from iSTAT, CHEM8, CG4+, or others. This also includes additional metabolic panel analysis, such as from a PICOLLO system; data from a GEM blood analyzer, and any other standard lab-based blood analyzer to measure any features in blood (such as freezing point osmometer or any biomarker analysis).

### a. Collective Organ Digital Twin

In some forms of the apparatuses, systems, and/or methods, prior to storing collected biological data, the organ digital twin contained donor data, point of care data, and/or one or more classes of biological data. Preferably, the data are collected from or obtained for one or more different instances of the organ of interest. Preferably, the organ digital twin containing data of the instance of the organ of interest and data collected from or obtained for one or more different instances of the organ of interest constitutes a collective organ digital twin.

### b. Individual Organ Digital Twin

In some forms of the apparatuses, systems, and/or methods, donor data, point of care data, and biological data stored in an organ digital twin is collected from or obtained for only the instance of the organ of interest. Preferably, the organ digital twin containing data of the instance of the organ of interest constitutes an individual organ digital twin.

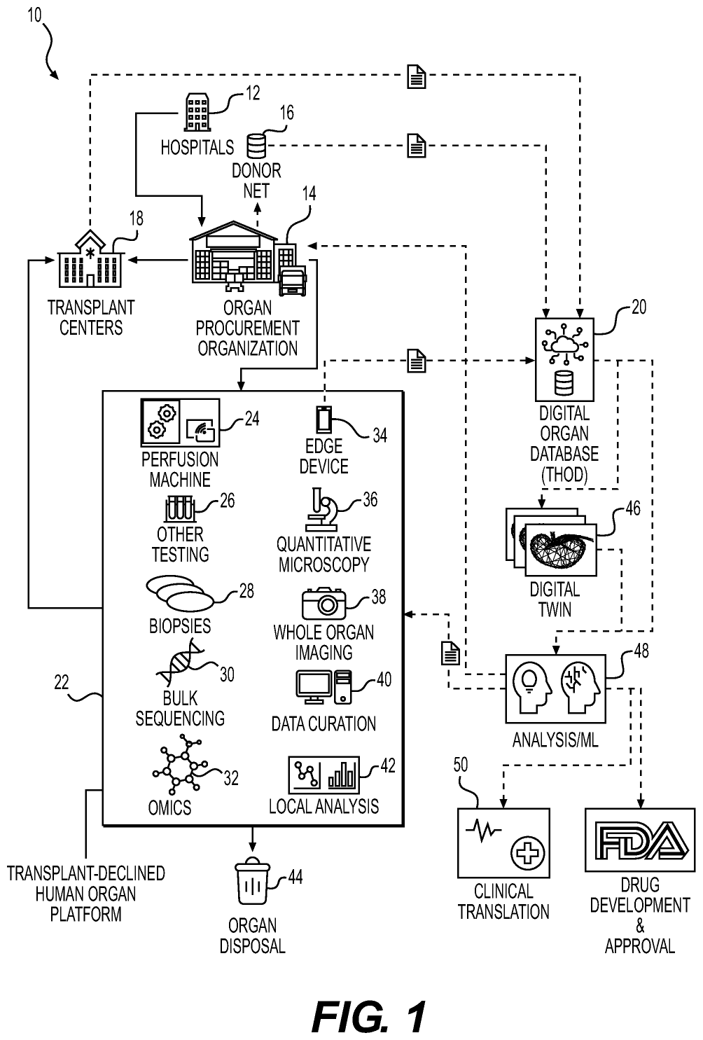

FIG. 1 is a non-limiting example of an organ digital twin ecosystem 10, according to aspects of the present embodiments. Digital twins (such as, organ digital twins) include computer models that provide digital replicas of individual, isolated failing and/or healthy organs. Using organ digital twins: real time identification of failure modes can be achieved, and predictions of appropriate responses to therapy can be made. In addition, organ digital twins may be used in connection with the present ecosystem of FIG. 1 as a drug development tool by allowing accelerated access to testing of human organs, thereby reducing the overall drug development timeframe and increasing the success rate. As is illustrated in FIG. 1, the organ digital twin ecosystem 10 includes a plurality of data streams (shown with dashed lines) as well as a framework for the management of physical organs (shown in sold lines).

Referring still to FIG. 1, the digital twin ecosystem 10 includes hospitals 12 which are a common source of organs that are being donated.

The organs may include lungs, hearts, kidneys, and livers as well as pancreases, stomachs, uteruses, thymuses, and intestines. The present disclosure discusses on lungs, hearts, kidneys, and livers, although the disclosed forms are also applicable to other organs, including those that are not transplantable and those that have yet to be successfully transplanted. Donated organs 12 are transported to the OPO or organ procurement organization 14, where they are assessed for viability. The donated organs can also be transported directly from the recovery hospital to the transplant center or the research center. This is especially the case for organs that have shorter periods of tolerable ischemia (e.g., hearts, livers). Organs that are approved for transplant are transported to transplant centers 18 to be matched with patients on one or more organ waiting lists. Organs that are declined for transplant may be deemed viable for research and may be transported to a center for research using the transplant-declined human organ (THOD) research platform 22. Once at the transplant-declined human organ platform 22, various forms of data and information may be captured from the organs via various machines, tools, methodologies, and systems including (but not limited to) perfusion machines 24, biopsies 28, bulk sequencing techniques 30, omics 30 (for example, techniques such as genomics, transcriptomics, metabolomics, proteomics, lipidomics, and/or single cell analyses, among other emerging technologies), quantitative microscopy 36, whole organ imaging 38, data curation 40, local data analysis 42, as well as other types of testing 26. In some forms, organs that have undergone additional testing and analysis on the transplant-declined human organ platform 22 may be deemed acceptable for transplant (for example, as a result of an error being identified in the initial organ viability assessment at the OPO 14, or due to additional data capture and analysis available on the transplant-declined human organ platform 22 but not available at the OPO 14). After organs that have been tested, measured, and analyzed on the transplant-declined human organ platform 22 are no longer viable, they may be discarded via organ disposal facilities 44.

Still referring to FIG. 1, all of the data captured on a given organ from the various sources available on the transplant-declined human organ platform 22 may be transmitted via a web-enabled device (such as an edge device) 34 that allows data to be uploaded to a digital organ database (or transplant-declined human organ database (THOD)) 20. For example, during ex vivo or in vivo (e.g. in situ) perfusion on a perfusion machine 24, functional organ data may be captured on the organ, via the device (such as an edge device), which may have more capability to acquire, store, and transmit the data than the built-in capabilities of the perfusion machine 24 itself. For example, the edge device also integrates data from multiple different local devices to stream them to a cloud-based database. During perfusion, the organ can also be exposed to therapies that are in various stages of research and trials, to see how the organ (i.e., a biologically active human organ) responds. The data, once on the digital organ database or THOD 20 can be stored, analyzed, and used to develop digital twins 46 of isolated organs. Importantly, once the digital twin 46 has been build, the digital twin 46 is preserved indefinitely even after the isolated organ upon which the digital twin 46 was based is disposed of or otherwise repurposed. Each digital twin 46 can then be used as part of a larger database of digital twins 46 for studies to see (via digital twin predictions) how various populations of organs, each with their own unique failure modes, will respond to a given therapy. The data, once on the digital organ database or THOD 20, can also be used to build more generalized organ models, and can be further analyzed via machine learning 48 to understand developing trends in organ health.

Referring still to FIG. 1, the analysis of the THOD 20 data (for example, by analyzing via machine learning multiple digital twins, each with a specific failure mode or modes associated therewith) can be used to guide clinical decision making (i.e., clinical translation 50) in the transplant context to identify when a drug is predicted to have a benefit on a patient and/or organ post-transplant. In addition, the data can be used to accelerate drug development (i.e., drug development and approval 52, shown in FIG. 1) in human organs for biotech/pharma applications via the full data capture/audit trail capabilities available via the device (such as an edge device) 34 and digital organ database (THOD) 20. In addition, analysis of the digital organ database data can be used both to inform failure mode analysis during perfusion 24 (or while an organ is undergoing other types of evaluation and analysis on the transplant-declined human organ platform 22) but also to inform assessment of individual organ transplant viability at the organ procurement organization 14 to bring more accurate decision-making to the viability assessment, thereby allowing an increased number of organs to be used for transplant. As a result, the methodology for determining if an organ can be used for transplant may be updated to reflect more accurate decision-making. Moreover, a third category of outcomes may be possible. Historically, organs were deemed to be either acceptable for transplant, or alternatively, those that were declined for transplant were deemed suitable for use in research applications only. With more informed decision-making from analysis of the digital organ database 20 data, organ procurement organizations 14 may determine that an organ falls into a third category where revitalization of an organ (for example, via various treatments on the transplant-declined human organ platform 22) is possible. Therefore, organs that are initially declined for transplant may no longer be re-purposed only for research; they may also be able to be revitalized and used for transplant.

Still referring to FIG. 1, demographic and donor history data (as well as other data about an individual donor) may be captured by the organ procurement organization 14, both with respect to transplanted organs as well as with respect to organs that are sent to the transplant-declined human organ platform 22 for research, testing, and/or revitalization. This demographic and donor history data is captured by the donor net 16 and similarly transmitted to the digital organ database 20 such that information about all donated organs may be captured and used for analysis. In addition, data regarding patient outcomes associated with organs that have been successfully (or unsuccessfully) transplanted is captured by the transplant centers 18 and similarly transmitted to the digital organ database 20 such that any learnings regarding patient outcomes can also be captured in the digital organ database 20 (THOD).

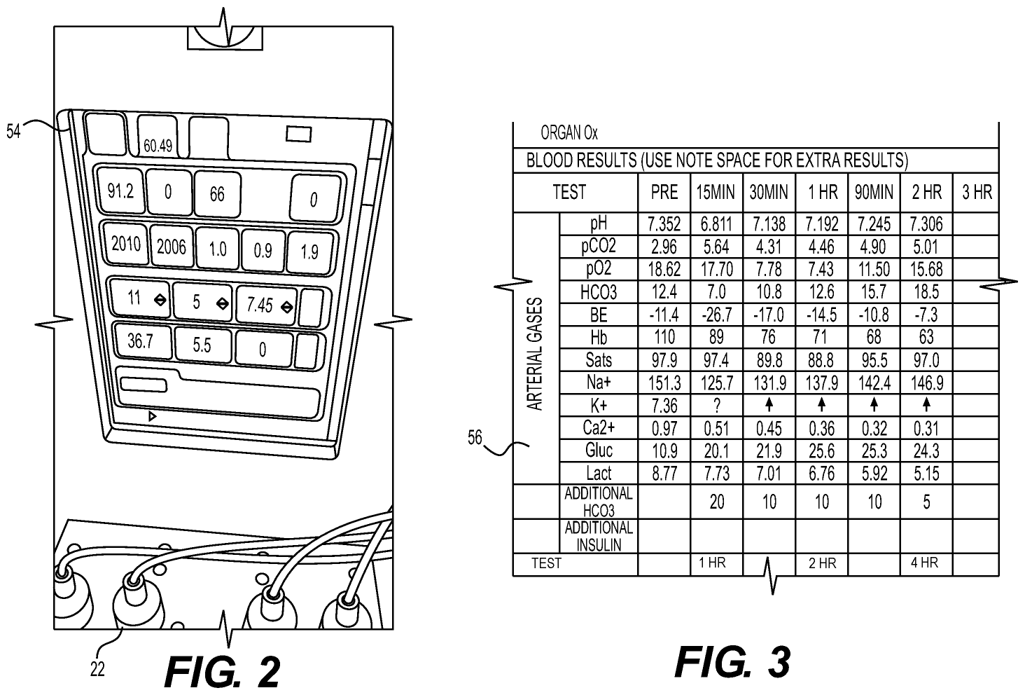

FIG. 2 illustrates a digital interface 54 on an exemplary perfusion machine 24. The data displayed on the digital interface 54 may be hard to access, may take a while to update, and is often only able to be logged or recorded via a manual data sheet 56, illustrated in FIG. 3. The manual data sheet 56 includes cross-outs, numbers that are hard to read, missing numbers, a question mark (?) in one case, and lack of resolution in several cases. In addition, the manual data sheet 56 contains only manual data, and no data that is in a digital format that is easy to be shared and/or accessed by others, or analyzed. Therefore, there are many challenges associated with data analysis and information sharing (and ultimately, breakthroughs and innovation) using the current machines and protocols.

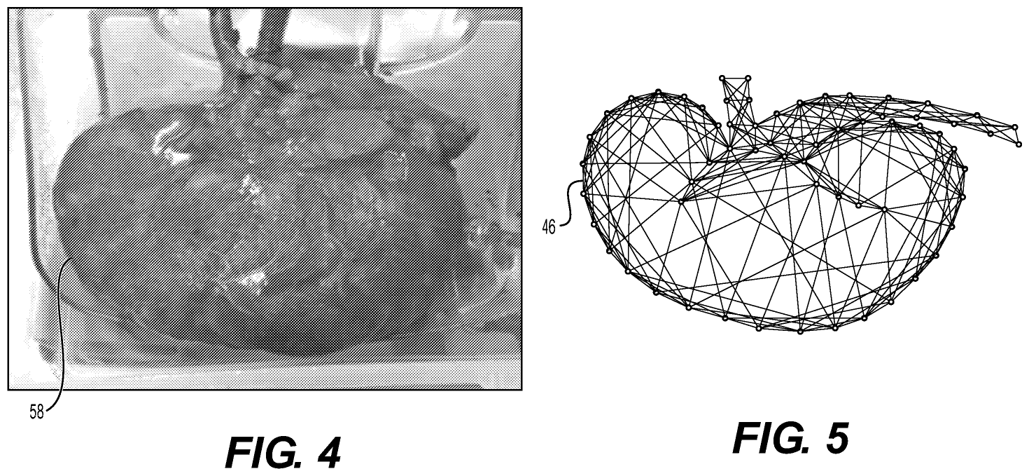

In contrast to the limitations of many of the perfusion and other types of machines being used today, the present disclosure details how a digital twin 46 (shown in FIG. 5) can be built based on an isolated organ 58 (shown in FIG. 4), the digital twin 46 being a digital replica of the isolated organ 58 that “lives on” (i.e., is retained indefinitely) even after the isolated organ 58 is no longer viable, such that further study and analysis can occur indefinitely using the digital twin 46. The digital twin 46 may include any or the following information: representation of individual failure mode(s) specific to the isolated organ 58, as well as demographic information from the donor, the donor's past health history, anatomic information about the isolated organ 58, functional data (for example, blood flow characteristics) of the isolated organ 58 that was captured during ex vivo perfusion 24, quantitative microscopy data 36, whole or partial organ imaging data 40, bulk DNA or RNA sequencing data 30 of the isolated organ 58, biopsy information 28, omics data 32 (i.e., genomic data, transcriptomic data, metabolomics data, proteomic data, lipidomics data, and/or single cell analyses data), treatment history information, and/or other types of data. Organs can remain viable during perfusion 24 for a period of about 5 days (120 hours) allowing (in some cases) organs to be revitalized. In addition, 5 days is often enough time to determine how an organ responds to various treatments. Time-based data can also be captured such that snapshots of the organ at the beginning, middle, and end can be compared in order to assess organ functionality over time, which in turn may lead to further insights on organ treatments and failure modes.

### c. Organ Perfusion Systems

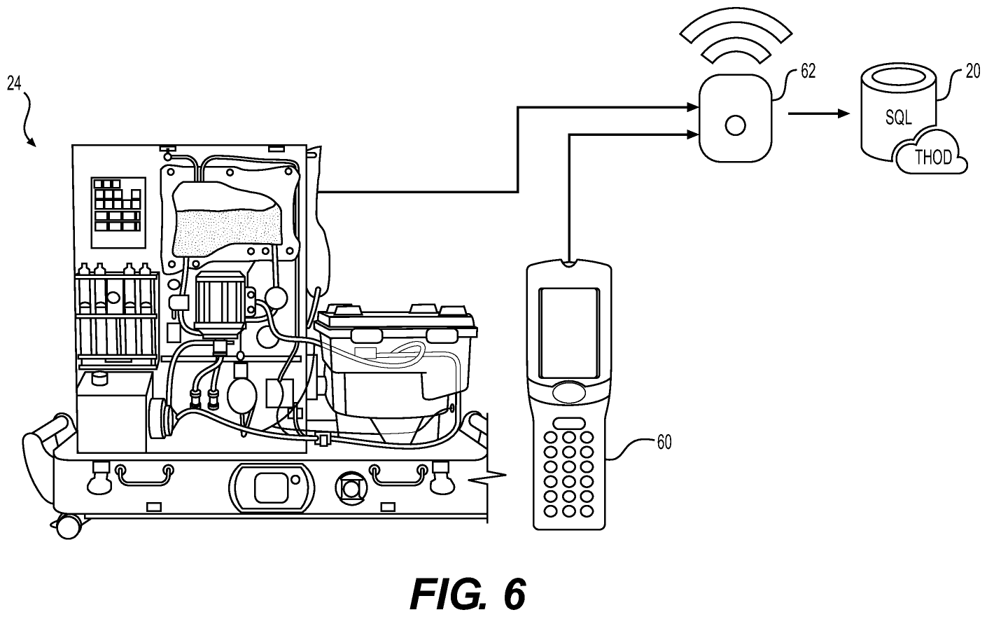

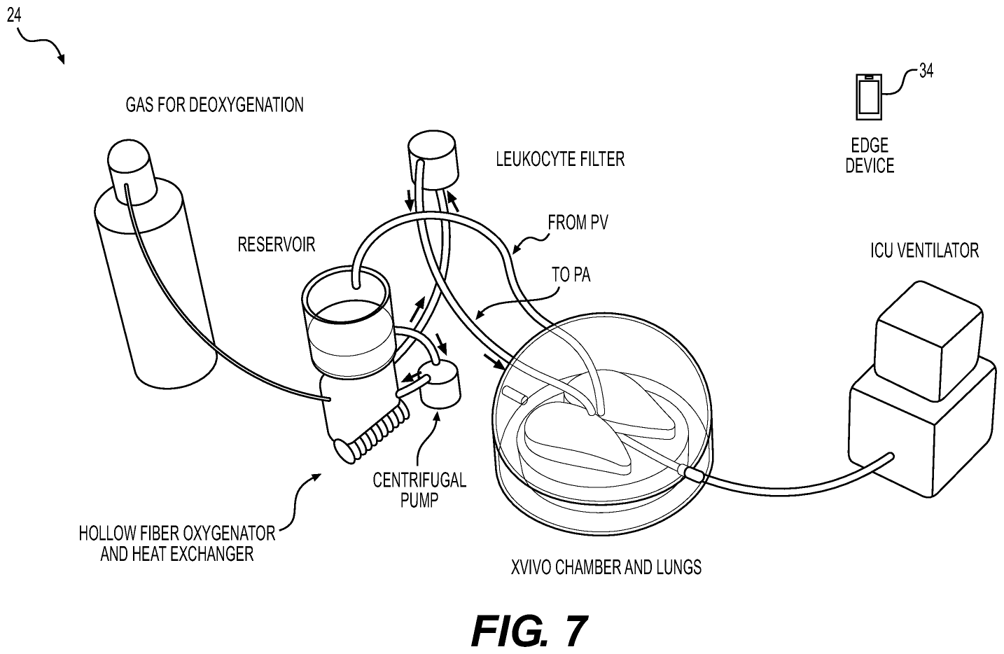

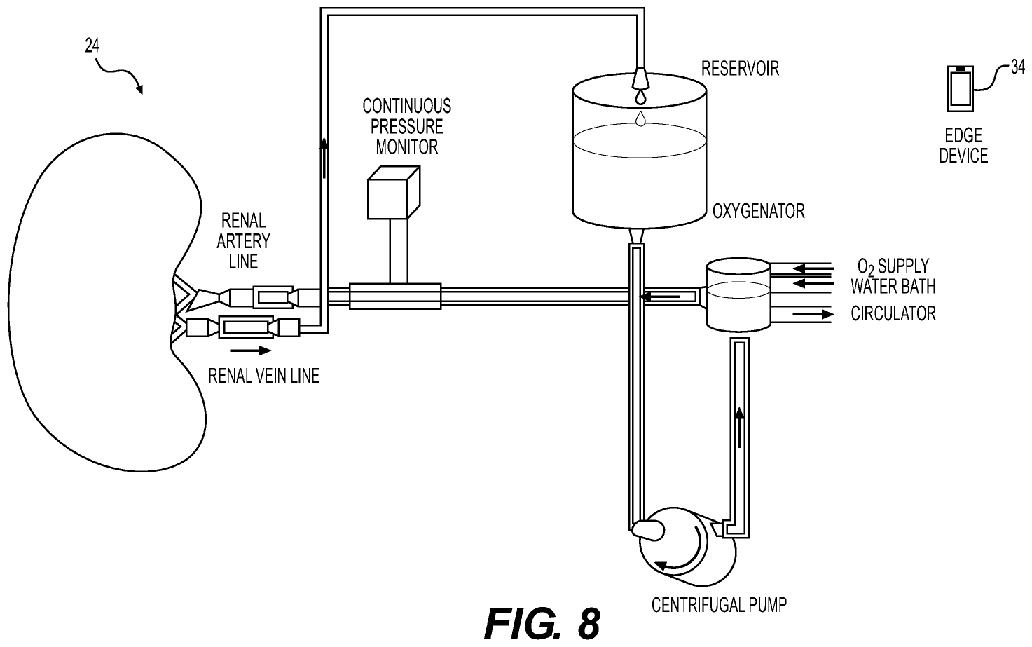

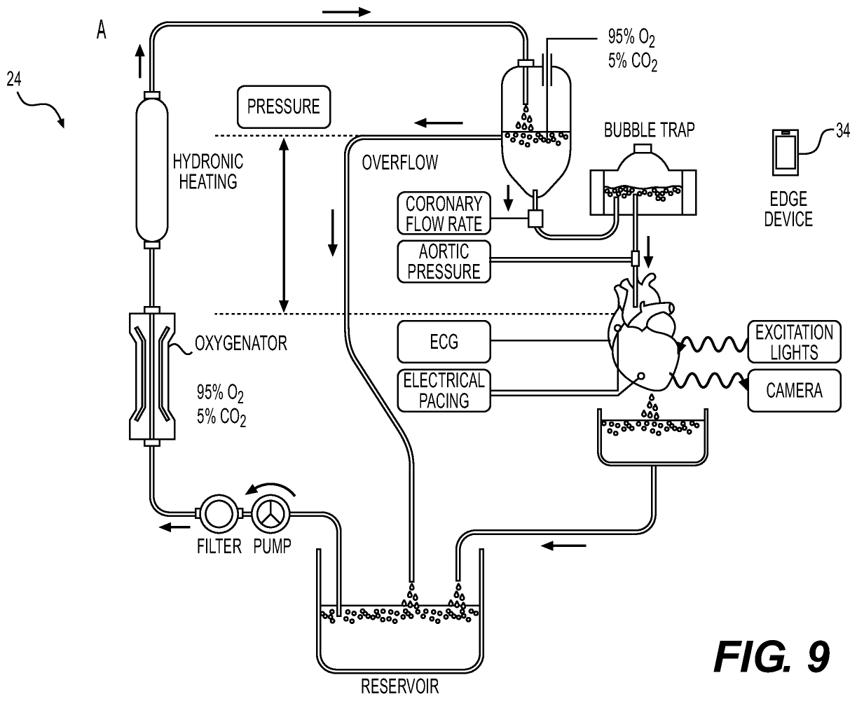

FIGS. 6-9 illustrate embodiments of perfusion machines 24 equipped with device (such as an edge device) 34, according to aspects of the present embodiments. FIG. 6 illustrates a liver being perfused. The system of FIG. 6 may include an iSTAT (or equivalent) digital interface 60 communicatively coupled to a router 62 which is equipped to transmit perfusion data to the digital organ database 20, which may be configured to run SQL, or other database management software. FIG. 7 illustrates lungs being perfused with a unitary device (such as an edge device) equipped to collect data and transmit it to the digital organ database 20. FIG. 8 illustrates a kidney being perfused with a unitary device (such as an edge device) equipped to collect data and transmit it to the digital organ database 20. FIG. 9 illustrates a heart being perfused with a unitary device (such as an edge device) equipped to collect data and transmit it to the digital organ database 20. In some forms, the present disclosure is applicable to organs that are still in situ within a human body. For example, tubes and sensors are fluidly and communicatively coupled to the organ in situ within a human body, the organ then being able to be bypassed such that relevant data and measurements can be captured to help guide treatment and clinical decision-making.

### d. Clinical Transplant Application

The workflow includes various graphical user interfaces of a clinical transplant application, which may be configured to run on a mobile device, personal computer or other electronic device, according to aspects of the present embodiments. In some embodiments, a mobile device running the clinical transplant application may interface with a device (such as an edge device) 34 (or perfusion machine 24 itself) such that the two are communicatively coupled and the mobile device is able to receive, display, log, record, and/or transmit data received from the perfusion machine 24 and/or device (such as an edge device) 34. In embodiments where there is no device (such as an edge device) present in the vicinity of the perfusion machine 24, a mobile device running the clinical transplant application may act as a device (such as an edge device) 34 by providing data entry fields where users can enter the data from the perfusion machine 24, which may subsequently be transmitted and uploaded to the digital organ database 20. Therefore, using the clinical transplant application of the present embodiments in connection with a multitude of modern day wifi-enabled electronic devices, any perfusion machine 24 can be transformed into one that is capable of capturing the necessary data to build a digital twin 46, even in the absence of a dedicated device (such as an edge device) 34.

### e. Data Storage

Preferably, biological data collected in the apparatuses, systems, and/or methods disclosed herein can be stored in computing platforms capable of supporting large amounts of data. These include, but are not limited to, cloud computing platforms such as Microsoft Azure, Amazon Web Services Cloud, Google Cloud, IBM Cloud, Oracle Cloud, Red Hat Cloud, Cloudways, etc. Preferably, these platforms host relational databases that store structured data for rapid retrieval and analysis of records.

### ii. Organ Management

In some forms, the apparatuses, systems, and/or methods further involve analyzing the data in the organ digital twin to determine the condition of the instance of the organ of interest. In some forms, the method is as described above, excerpt that an alert is generated if the condition of the instance of the organ of interest determined by the analysis of the organ digital twin indicates that the instance of the organ of interest needs mechanical intervention, therapeutic intervention, or both. In some forms, the method further involves altering the ex vivo or in vivo (e.g. in situ) perfusion conditions for the instance of the isolated organ of interest based on the condition of the instance of the organ of interest determined by the analysis of the organ digital twin. The condition(s) described here can be a healthy state, a disease, disorder, or defect identified in the organ of interest.

In some forms, the apparatuses, systems, and/or methods further involve performing a mechanical intervention on the instance of the organ of interest based on the condition of the instance of the organ of interest determined by the analysis of the organ digital twin.

In some forms, the apparatuses, systems, and/or methods further involve performing a therapeutic intervention (such as treatment) on the instance of the organ of interest based on the condition of the instance of the organ of interest determined by the analysis of the organ digital twin to rehabilitate the instance of the organ of interest.

### iii. Modes of Failure

Also included in the apparatuses, systems, and/or methods is a platform for assessing a failure mode of an isolated organ, the platform containing: an organ perfusion machine; and a device (such as an edge device) communicatively coupled to the organ perfusion machine, the device (such as the edge device) capturing functional data from the perfusion machine during perfusion of the isolated organ, wherein the functional data of the isolated organ is used to determine at least one failure mode of the isolated organ.

In some forms, the platform includes a digital organ database communicatively coupled to a device (such as an edge device), the digital organ database configured to create and store a digital twin of the isolated organ.

In some forms, the apparatuses, systems, and/or methods further involve analyzing biological data in the organ digital twin to determine one or more modes of failure of the instance of the organ of interest. In some forms, the method further involves performing a therapeutic intervention (such as treatment), ex vivo and/or in vivo (e.g., in situ), on the instance of the organ of interest based on one or more of the determined modes of failure. Generally, analyzing the data can identify a logical connection between one or more modes of organ failure to one or more forms of therapeutic intervention. Generally, the determined modes of failure are in one or more classes of modes of failure. Classes of modes of failure include, but are not limited to, one or more of perfusion device failure modes, vascular failure modes, metabolic failure modes, immunological failure modes, and surgical failure mode (such as hemorrhagic failure).

Perfusion device failure modes include, but are not limited to, dialysis failure. Dialysis failure can be detected from one or more of hypotension, hemorrhage, low hematocrit, low pH, high potassium, given that dialysis helps to normalize one or more of these factors.

Vascular failure modes include, but are not limited to, one or more of arterial or venous hypertension or hypotension (non-device related), edema, microvascular obstruction.

Metabolic failure modes include, but are not limited to, one or more of lactic acidosis, metabolic alkalosis, respiratory acidosis, respiratory alkalosis, succinate-mediated electron transport disruption.

Immunological failure modes include, but are not limited to, one or more of dysfunctional regulated cell death, dysfunctional IL-1 or TNF-mediated inflammation, excessive damage associate molecular pattern release (e.g., HMGB1, cell free DNA, ATP, uric acid).

It should be noted that abnormal values for some of the factors described above (arterial/venous hypotension, hemorrhage, low hematocrit, low pH, high potassium, given that dialysis helps to normalize one or more of these factors) can also arise from excessive cell death due to immunological failure. Thus, although the failure modes are described individually herein, overlaps between the parameters of failure modes can also exist, such that multiple failure modes can be present simultaneously.

### iv. Drug Testing and Pharmacokinetics

In some forms, the apparatuses, systems, and/or methods further involve performing a therapeutic intervention (such as treatment) on the instance of the organ of interest with a proposed therapy. In some forms, the apparatuses, systems, and/or methods further involve collecting additional biological data from the instance of the organ of interest and storing the collected additional biological data in the organ digital twin. In some forms, the apparatuses, systems, and/or methods further involve analyzing the data in the organ digital twin to determine one or more of the effects of the proposed therapy on the instance of the organ of interest. In some forms, the apparatuses, systems, and/or methods further involve performing a therapeutic intervention (such as treatment) on the instance of the organ of interest with a proposed therapy, collecting additional biological data from the instance of the organ of interest, storing the collected additional biological data in the organ digital twin, and analyzing the data in the organ digital twin to determine one or more of the effects of the proposed therapy on the instance of the organ of interest. In some forms, the apparatuses, systems, and/or methods further involve repeating the treating, collecting, storing, analyzing, and determining steps with a second proposed therapy. In some forms, the apparatuses, systems, and/or methods further involve storing the determined effects in the organ digital twin.

In some forms, the apparatuses, systems, and/or methods further involve analyzing the data in the organ digital twin to predict one or more effects on the organ of interest of a therapy of interest. In some forms, the method further involves treating the instance of the organ of interest with the therapy of interest, collecting additional biological data from the instance of the organ of interest, storing the collected additional biological data in the organ digital twin, and analyzing the data in the organ digital twin to determine one or more of the effects of the therapy of interest on the instance of the organ of interest.

In some forms, the apparatuses, systems, and/or methods further involve analyzing the biological data in the organ digital twin to predict one or more effects on the organ of interest of a therapy of interest. In some forms, the apparatuses, systems, and/or methods further involve treating the instance of the organ of interest with the therapy of interest, collecting additional biological data from the instance of the organ of interest, storing the collected additional biological data in the organ digital twin, and analyzing the data in the organ digital twin to determine one or more of the effects of the therapy of interest on the instance of the organ of interest. In some forms, the apparatuses, systems, and/or methods further involve repeating the treating, collecting, storing, analyzing, and determining steps with a second therapy of interest.

In some forms, the apparatuses, systems, and/or methods are as described above, except that they further involves storing the determined effects of the therapy of interest in the organ digital twin.

### v. Transplant Recipient

In some forms, the apparatuses, systems, and/or methods further involve integrating transplant recipient data with all or a portion of the data contained in the organ digital twin and/or all or a portion of data derived from the organ digital twin.

In some forms, the apparatuses, systems, and/or methods further involve analyzing the integrated transplant recipient data and organ digital twin data to assess suitability of the instance of the organ of interest for transplant into the recipient.

### vi. Real-Time Visualization of Data

In some forms, the apparatuses, systems, and/or methods further involve transmitting and/or displaying, in real time and/or as a static record, all or a portion of the data contained in the organ digital twin and/or all or a portion of data derived from the organ digital twin. In some forms, the data is transmitted, wirelessly or via a wired network, to one or more remote devices. In some forms, the displaying involves displaying the transmitted data on the remote device.

In some forms, the data derived from the organ digital twin contains the condition of the instance of the organ of interest, a generated alert, altered ex vivo or in vivo (e.g. in situ) perfusion conditions, a mechanical intervention, a therapeutic intervention, and/or actions or interventions suggested by the analysis of data in the organ digital twin. The condition(s) described here can be a healthy state, a disease, disorder, or defect identified in the organ of interest.

In some forms, the apparatuses, systems, and/or methods further involve analyzing all or a portion of the data contained in the organ digital twin to produce derivatized data from the organ digital twin.

In some forms, the apparatuses, systems, and/or methods further involve modeling responses of an organ of interest, including analyzing the response of an organ digital twin, described herein, to an action of interest.

In some forms, the action of interest is a change in one or more of the data contained in the organ digital twin and/or in one or more of the data derived from the organ digital twin.

In some preferred forms, the organ digital twin is stored in a digital physical medium. In some forms, the data is stored by writing the data in a digital physical medium. Preferably, the storage is performed by a computer configured to accomplish the storage.

### vii. Machine Learning Module

With rapid technological advances, especially in high-performance central processing units and graphics processing units, artificial intelligence is increasingly being explored in medical diagnosis applications. These advances provide an opportunity to develop platforms to collect digital interfaces to collect, process, store, and/or display digital data. With the availability of these digital data, machine learning modules can be incorporated into these digital platforms, to provide analytics in organ donor settings.

Accordingly, in some forms, the apparatuses, systems, and/or methods further involve transmitting all or a portion of the data contained in the organ digital twin and/or all or a portion of data derived from the organ digital twin, and using a machine learning module to compute the current health state for the instance of the organ of interest.

In some forms, the machine learning module is configured to train a machine learned model based on the transmitted data. In some forms, the transmitted data includes donor data, point of care data, and/or one or more classes of biological data described herein.

In some forms of the machine learning module, computing the current health state for the organ of interest involves using enriched data. In some forms, the enriched data is from the same type of organ from the same donor or different donors.

In some forms, the machine learning module is configured to train a machine learned neural network model, a Bayesian model, an artificial intelligence system, a rules-based system, or a combination thereof. In some forms, the machine learning module trains the models in a supervised, unsupervised, or semi-supervised manner.

In some forms, the machine learning module contains neural networks selected from recurrent neural networks, convolutional neural networks, and artificial neural networks.

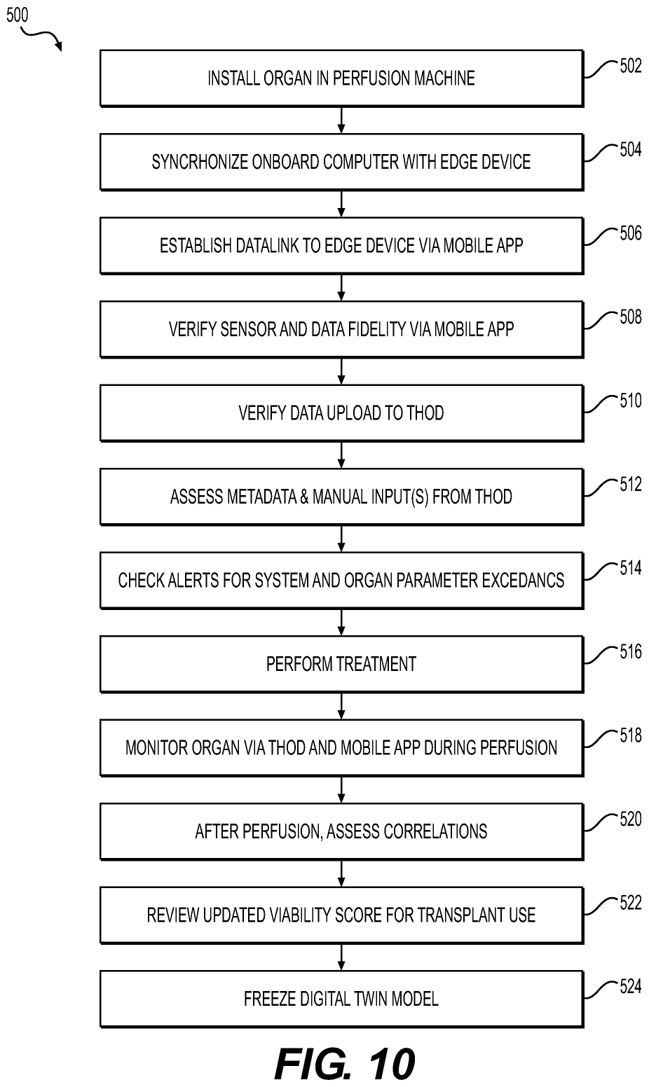

FIG. 10 illustrates a method 500 of creating an organ digital twin 46, according to aspects of the present embodiments.

By providing data capture, digitization, data transmission and data analysis capabilities to conventional organ treatment equipment such as perfusion machines, the disclosed apparatuses, systems, and methods provide means for breakthroughs in organ-related patient outcomes and drug development.

Each of the instruments, devices, and sensors described in the present disclosure may include a wired power supply or a wireless power supply such as a battery, capacitor, or other suitable mechanism.

Elements of different implementations described may be combined to form other implementations not specifically set forth previously. Elements may be left out of the processes described without adversely affecting their operation or the operation of the system in general. Furthermore, various separate elements may be combined into one or more individual elements to perform the functions described in this specification.

Some specific apparatuses, systems, and/or methods are described below.

A method of creating an organ digital twin, the method involving:
installing an isolated organ within a perfusion machine;
communicatively coupling the perfusion machine to an edge device;
communicatively coupling the edge device to a digital organ database;
capturing functional data from the isolated organ during perfusion, the functional data being transmitted to the digital organ database via the edge device;
assessing at least one failure mode of the isolated organ; and
freezing the organ digital twin upon completion of perfusion of the isolated organ.

A platform for assessing a failure mode of an isolated organ, the platform containing:
an organ perfusion machine; and
an edge device communicatively coupled to the organ perfusion machine, the edge device capturing functional data from the perfusion machine during perfusion of the isolated organ,
wherein the functional data of the isolated organ is used to determine at least one failure mode of the isolated organ. In some forms, the platform contains at least one additional data source for capturing data on the isolated organ, the at least one additional data source containing equipment for capturing at least one of: quantitative microscopy data, whole organ imaging data, partial organ imaging data, bulk DNA sequencing data, bulk RNA sequencing data, biopsy information, genomic data, transcriptomic data, metabolomics data, proteomic data, lipidomics data, and single cell analyses data. In some forms, the platform contains a digital organ database communicatively coupled to the edge device, the digital organ database configured to create and store a digital twin of the isolated organ.

A clinical transplant application for an electronic device, the clinical transplant application containing at least one graphical user interface for inputting functional organ data from a perfusion machine,

wherein the clinical transplant application is communicatively coupled to a digital organ database and configured to transmit the functional organ data to the digital organ database.

In recent years, Digital Twin technology has also been applied to healthcare, with a focus on creating digital twins of human organs to recapitulate key physiological phenomena that directly inform clinical decision-making. In the field of precision cardiology, for example, electrocardiogramuclear, and ultrasound imaging data is used to create an anatomically accurate, electrophysiological mathematical model of the heart (G. Coorey, G. A. Figtree, D. F. Fletcher, V. J. Snelson, S. T. Vernon, D. Winlaw, S. M. Grieve, A. McEwan, J. Y. H. Yang, P. Qian, K. O′Brien, J. Orchard, J. Kim, S. Patel, and J. Redfern, “The health digital twin to tackle cardiovascular disease-a review of an emerging interdisciplinary field,” npj Digital Medicine, vol. 5, pp. 1-12, August 2022). This cardiac Digital Twin can then be used to simulate a patient's response to different therapies by incorporating data derived from previous patients and/or animal experiments (G. Coorey, G. A. Figtree, D. F. Fletcher, V. J. Snelson, S. T. Vernon, D. Winlaw, S. M. Grieve, A. McEwan, J. Y. H. Yang, P. Qian, K. O'Brien, J. Orchard, J. Kim, S. Patel, and J. Redfern, “The health digital twin to tackle cardiovascular disease—a review of an emerging interdisciplinary field,” npj Digital Medicine, vol. 5, pp. 1-12, August 2022). However, the application of Digital Twin technology in healthcare is not without its challenges. Obtaining the data required to create an accurate and effective model of a patient's physiology is difficult, as all data used for Digital Twin construction must be obtained noninvasively. This naturally favors applications that use imaging data to reconstruct patient anatomy and/or physiology, but many physiological processes are difficult to obtain in greater detail than simple metrics from blood tests. Furthermore, the workflow of the healthcare system is not suited for continuous data collection, which further complicates the process of creating a Digital Twin of a patient's physiology.

The challenges of applying Digital Twin technology in healthcare highlight the potential benefits of using this approach in the ex vivo transplant organ perfusion space or other applications involving in vivo (e.g. in situ) organ perfusion. During normothermic machine perfusion (NMP), an organ is accessible to various forms of data collection that are not possible while it is inside a living patient. By developing automated data acquisition systems, this data can be obtained continuously without disrupting the organ's health or further burdening the healthcare team. Consequently, there is practical value in constructing a Digital Twin of an organ on NMP and using mathematical modeling to predict the potential benefit or risk associated with different interventions. Such mathematical models are discussed in further detail below.

### viii. Theoretical Mathematical Models for Real-Time Assessment of Organ Pathology in the Future

Real time physiological data acquisition technologies may aid transplant surgeons in diagnosing failure modes of transplant organs on normothermic machine perfusion. However, the relationships between physiological variables acquired in real time is complex and parsing out the nature of the organ's pathology by monitoring each signal individually may result in erroneous diagnoses and clinical decisions. To augment the utility of the real-time data acquisition system, it can be beneficial to develop mathematical models of the organ's physiology that may provide insight into the organ's pathology. Outlined are the types of mathematical models that can contribute meaningful information to the clinical decision-making process in the context of transplant organs on NMP. For non-limiting illustrative purposes, the initial focus is on applying these modeling strategies to diagnose failure modes in kidneys on NMP, with the understanding that these modeling strategies can also be applied to other organs such as the heart, lung and liver. For each model, the basic mathematical structure is described followed by a discussion of the model's utility for diagnosing organ failure modes in real time.

The kidney volume is composed of multiple interacting tissue compartments, each with different transport and physiological properties. During NMP, the vascular space fills with the arterial flow driven by the perfusion system. Part of this flow is lost to filtration at the glomerulus. The filtered volume enters the tubular space where it is either reabsorbed back into the vascular space, or excreted as urine. This structural organization naturally lends itself to compartmental modeling of volume flux between the different renal tissues, wherein a system of ordinary differential equations defines the rate of change of the volume in each compartment:

$$dV / dt = Inflow - Outflow - GFR + Rebsorption - Leak \qquad ( 2.1 )$$

$$dT / dt = GFR - Reabsorption - UrineExeretion \qquad ( 2.2 )$$

For V, the volume of fluids in the vascular compartment and T, the fluid volume in the tubular compartment. This modeling methodology is straightforward and physiologically meaningful, as it is directly applicable to data that is readily acquired with a real time data acquisition system (arterial and venous flows and urine output). Furthermore, this modeling strategy can be used to estimate microvascular leak (“Leak” in equation 1), wherein volume escapes the vascular space into the parenchyma, causing edema and an increased kidney volume. The change in kidney mass over time can be represented as a sum of the change of the volume in the vascular and tubular compartments:

$$dM / dt = ρ ( dV / dt + dT / dt ) \qquad ( 2.3 )$$

For M, mass of the kidney and ρ, the density of water (1 g/ml). Substituting equations 1 and 2 for the derivatives of the vascular and tubular volumes and rearranging equation 3, gives:

$$Leak = Inflow - Outflow - UrineExeretion - ( \frac{1}{ρ} ) \frac{dM}{dt} \qquad ( 2.4 )$$

Thus, with a sufficiently accurate measure of the kidney mass, arterial and venous flows, and urine excretion, this simplified modeling strategy could be used to estimate the rate of microvascular leak in the kidney. However, this simplified model does not take into account the volume of fluid entering and exiting the interstitial compartment. A more accurate model may introduce dI/dt to the set of equations described above for I, the volume of fluids in the interstitial compartment. However, decoupling the interstitial compartment from other compartments would not be achievable with current set of sensors in the perfusion circuit. Using additional sensors such as probes that can measure osmolarity in the interstitium or using cross sectional imaging to gather information about arteries, veins, and collecting system, a more representative compartmental model can be created.

Of significant importance to the assessment of the physiology of an organ on NMP are the hemodynamic variables that are monitored by the perfusion system which include, at a minimum, arterial flow and pressure. From a practical standpoint, these variables must be monitored to assess the quality of the perfusion of the organ, as this will have important implications for the organ's health during NMP. Additionally, these measurements lend themselves to hemodynamic modeling using Windkessel models. Windkessel models were originally developed and used to estimate blood transport within the heart and the greater cardiovascular system. These studies model connected vascular spaces using electrical circuit theories such as Ohm's Law, wherein a pressure drop P across a resistor is equal to the product of the flow (Q) and resistance (R):

$$P = QR \qquad ( 2.5 )$$

And the flow across a capacitor is equal to the product of the capacitance, C and the temporal change in pressure:

$$Q = C \frac{dP}{dt} \qquad ( 2.6 )$$

A 4-element Windkessel model, assumes that a vascular system can be modeled as a resistor (jagged line) in series with a compliant preglomerular vascular space (modeled as a resistor and capacitor in parallel), in series with a third resistor. Previous studies have used this Windkessel model to estimate glomerular pressure (PGlom) by fitting the model to renal arterial pressure and flow data obtained in living patients. By fitting these semi-continuous data to a Windkessel model, the investigators were able to estimate afferent resistance RAff, efferent resistance REff, and the afferent compliance/capacitance CAff in addition to glomerular pressure (D. Collard, P. M. van Brussel, L. van de Velde, G. W. Wijntjens, B. E. Westerhof, J. M. Karemaker, J. J. Piek, J. A. Reekers, L. Vogt, R. J. de Winter, and B. -J. H. van den Born, “Estimation of Intraglomerular Pressure Using Invasive Renal Arterial Pressure and Flow Velocity Measurements in Humans,” Journal of the American Society of Nephrology: JASN, vol. 31, pp. 1905-1914 August 2020; P. Segers, E. R. Rietzschel, M. L. De Buyzere, N. Stergiopulos, N. Westerhof, L. M. Van Bortel, T. Gillebert, and P. R. Verdonck, “Three- and four-element Windkessel models: assessment of their fitting performance in a large cohort of healthy middle-aged individuals,” Proceedings of the Institution of Mechanical Engineers. Part H, Journal of Engineering in Medicine, vol. 222, pp. 417-428, May 2008). They showed that these parameters (primarily glomerular pressure) varied between patients with diabetes and healthy controls which provides insight into mechanisms of diabetic hyperfiltration (D. Collard, P. M. van Brussel, L. van de Velde, G. W. Wijntjens, B. E. Westerhof, J. M. Karemaker, J. J. Piek, J. A. Reekers, L. Vogt, R. J. de Winter, and B. -J. H. van den Born, “Estimation of Intraglomerular Pressure Using Invasive Renal Arterial Pressure and Flow Velocity Measurements in Humans,” Journal of the American Society of Nephrology: JASN, vol. 31, pp. 1905-1914 August 2020; P. Segers, E. R. Rietzschel, M. L. De Buyzere, N. Stergiopulos, N. Westerhof, L. M. Van Bortel, T. Gillebert, and P. R. Verdonck, “Three- and four-element Windkessel models: assessment of their fitting performance in a large cohort of healthy middleaged individuals,” Proceedings of the Institution of Mechanical Engineers. Part H, Journal of Engineering in Medicine, vol. 222, pp. 417-428, May 2008). In the context of kidneys on NMP, these parameters could provide insight into the vasoreactivity of the kidney's vasculature, the kidney's vascular compliance and its glomerular function. These parameters may correlate with clinical and/or pathological outcomes, and may be indicators of vascular dysfunction or obstruction which have been characterized previously (J. R. DiRito, S. A. Hosgood, M. Reschke, C. Albert, L. G. Bracaglia, J. R. Ferdinand, B. J. Stewart, C. M. Edwards, A. G. Vaish, S. Thiru, D. C. Mulligan, D. J. Haakinson, M. R. Clatworthy, W. M. Saltzman, J. S. Pober, M. L. Nicholson, and G. T. Tietjen, “Lysis of cold-storage-induced microvascular obstructions for ex vivo revitalization of marginal human kidneys,” American Journal of Transplantation, vol. 21, no. 1, pp. 161-173, 2021).

The third general form of modeling that may provide insights into the health of a kidney on NMP are general physiological modeling. Physiological models are collections of mathematical equations to describe a physiological process that typically involves transport of fluid and/or solutes or gases across a biologically relevant membrane. In the case of the kidney, we focus on the physiological processes of glomerular filtration, tubular reabsorption, and oxygen consumption, all of which are both relevant to kidney physiology and potentially integrable in the NMP workflow.

For example, using point-of-care blood and urine gas measurement device, it is possible to estimate GFR and tubular reabsorption rate in a kidney on NMP. By fitting previously developed models of glomerular filtration (W. M. Deen, B. Satvat, and J. M. Jamieson, “Theoretical model for glomerular filtration of charged solutes,” The American Journal of Physiology, vol. 238, pp. F126-139, February 1980) and tubular reabsorption (A. T. Layton, A. Edwards, and V. Vallon, “Adaptive changes in GFR, tubular morphology, and transport in subtotal nephrectomized kidneys: modeling and analysis,” American Journal of Physiology. Renal Physiology, vol. 313, pp. F199-F209, August 2017; A. T. Layton and H. E. Layton, “A computational model of epithelial solute and water transport along a human nephron,” PLOS computational biology, vol. 15, p. e1006108, February 2019) to this data, the emerging parameters may provide insight into the physiological function of the kidney. Perhaps most relevant to the NMP context are models of oxygen transport and consumption, which are readily integrated into the NMP setting through measurement of oxygen saturation in the arterial and venous lines. For Sa and Se, the arterial and venous oxygen saturation, respectively, the oxygen saturation along the idealized length of the kidney can be modeled using a simplified Krogh cylinder model (J. L. Zhang, G. Morrell, H. Rusinek, L. Warner, P. -H. Vivier, A. K. Cheung, L. O. Lerman, and V. S. Lee, “Measurement of renal tissue oxygenation with blood oxygen level-dependent MRI and oxygen transit modeling,” American Journal of Physiology—Renal Physiology, vol. 306, pp. F579-F587, March 2014; D. Goldman, “Theoretical Models of Microvascular Oxygen Transport to Tissue,” Microcirculation (New York, N.Y.: 1994), vol. 15, pp. 795-811, November 2008).

As such, perfusion experiments incorporating arterial as well as venous pressure, flow, and oxygenation measurement can utilize such mathematical models for real-time analysis of organ pathophysiology in the future.

### EXAMPLES

### Example 1: Scalable System and Method for Real-Time Monitoring of Ex Vivo Organ Perfusion Metrics

The field of normothermic ex vivo organ perfusion has evolved substantially over the last decade with the development of new technologies that can preserve organs for extended periods of time (J. Noll, S. Beecham, and D. Seichter, “A Qualitative Study of Open Source Software Development: The Open EMR Project,” in 2011 International Symposium on Empirical Software Engineering and Measurement, pp. 30-39, September 2011. ISSN: 1949-3789). This extended preservation of the organ in a semi-physiological state allows for both organ physiologic assessment and testing therapeutic strategies outside the human body (G. T. Tietjen, S. A. Hosgood, J. DiRito, J. Cui, D. Deep, E. Song, J. R. Kraehling, A. S. Piotrowski-Daspit, N. C. Kirkiles-Smith, R. Al-Lamki, S. Thiru, J. A. Bradley, K. Saeb-Parsy, J. R. Bradley, M. L. Nicholson, W. M. Saltzman, and J. S. Pober, “Nanoparticle targeting to the endothelium during normothermic machine perfusion of human kidneys,” Science Translational Medicine, vol. 9, p. eaam6764, November 2017. Publisher: American Association for the Advancement of Science; C. Albert, L. Bracaglia, A. Koide, J. DiRito, T. Lysyy, L. Harkins, C. Edwards, O. Richfield, J. Grundler, K. Zhou, E. Denbaum, G. Ketavarapu, T. Hattori, S. Perincheri, J. Langford, A. Feizi, D. Haakinson, S. A. Hosgood, M. L. Nicholson, J. S. Pober, W. M. Saltzman, S. Koide, and G. T. Tietjen, “Monobody adapter for functional antibody display on nanoparticles for adaptable targeted delivery applications,” Nature Communications, vol. 13, p. 5998 October 2022. Number: 1 Publisher: Nature Publishing Group). However, there is currently a lack of tools for high-throughput monitoring and analysis of organs undergoing perfusion. Such tools would allow researchers to assess the health and functionality of organs in real-time, which is critical for developing new treatments and therapies for various diseases.

In this study, medical-grade perfusion devices from Medtronic as well as non-medical grade devices were utilized for measuring weight for the organ perfusion circuit. However, these devices typically do not have the ability to output data continuously or interface with other components. To solve this problem, an edge device was developed. Edge device is a term used in the fields of computer networking and embedded systems referring to any piece of hardware that controls data flow at the boundary between two networks (K. Cao, Y. Liu, G. Meng, and Q. Sun, “An Overview on Edge Computing Research,” IEEE Access, vol. 8, pp. 85714-85728, 2020; S. Dey, A. Mukherjee, H. S. Paul, and A. Pal, “Challenges of Using Edge Devices in IoT Computation Grids,” in 2013 International Conference on Parallel and Distributed Systems, pp. 564-569, December 2013. ISSN: 1521-9097).

This example describes the development of an edge device for communication between medical-grade perfusion devices and a centralized database. The edge device was built using a Raspberry Pi loaded with applications coded in Python that can continuously interpret various perfusion metrics including arterial flow, pressure, pump speed, organ weight, urine/bile production, oxygen saturation, and hematocrit. furthermore, the edge device was designed to be cost-effective, modular, and open-source, allowing for easy expansion in the future.

This example also describes the schema for a modular and scalable relational database used to store and analyze the continuous stream of data captured by the edge device. Relational databases are a popular choice for data storage and analysis due to their ability to efficiently manage large amounts of structured data (R. Kimball and M. Ross, The Data Warehouse Toolkit: The Complete Guide to Dimensional Modeling. John Wiley & Sons, August 2011. Google-Books-ID: XoS2oy1IcB4C; G. Manogaran, C. Thota, D. Lopez, V. Vijayakumar, K. M. Abbas, and R. Sundarsekar, “Big Data Knowledge System in Healthcare,” in Internet of Things and Big Data Technologies for Next Generation Healthcare (C. Bhatt, N. Dey, and A. S. Ashour, eds.), Studies in Big Data, pp. 133-157, Cham: Springer International Publishing, 2017). However, as data volumes increase, traditional relational databases may struggle to keep up with the demands of modern applications. To ensure that the database can handle the increasing amount of data required for high-throughput human organ research, a scalable schema is highly important. In this work, Azure SQL (Structured Query Language) cloud database was implemented to modify and adapt the star schema for ex vivo organ research.

Building on the scalable schema for ex vivo organ perfusion data storage, this work incorporated the use of interactive data visualization tools to display and analyze data from the relational database in real-time, as data is captured during ex vivo organ perfusion.

Overall, these examples highlight the potential of utilizing (1) open source hardware and software to capture data, (2) scalable structures for relational data storage, and (3) interactive data visualization tools to display and analyze perfusion data in real-time. These developments have the potential to revolutionize the field of normothermic ex vivo or in vivo (e.g. in situ) organ perfusion by allowing cost-effective and scalable monitoring systems that could lead to a better understanding of organ pathophysiology and development of new therapies. To demonstrate the utility of this monitoring system, arterial histamine was administered to a porcine kidney, and the porcine kidney's response was observed through the various sensors incorporated in the system.

### Materials and Methods

i. System for Continuous and Modular Data Capture During Ex Vivo Normothermic Organ Perfusion

For the ex vivo normothermic perfusion of kidneys, data pertaining to the hemodynamics of the kidney were collected and monitored: kidney mass, urine output, arterial flow, arterial pressure, pump speed, hematocrit, and arterial oxygen saturation. To accomplish this, ZP-50N digital force gauges by Baoshishan were used to measure kidney mass and urine output; a Medtronic Bio-console device to measure flow, pressure, and pump speed; and a Medtronic Bio-trend device to measure hematocrit and oxygen saturation.

In order for the sensors to connect to the Raspberry Pi, UOTEK USB to RS-232 Converter USB V2.0 cables were utilized, as serial drivers were required to establish connection. To connect the prior cables to the Medtronic devices, Agilent RS232-61601 cables were required, with an additional female-to-male serial cable “gender changer” utilized for the Bio-trend device.

Data collection from these sensors was standardized at a rate of 0.2 Hz (approximately a data point every five seconds) and integrated via an open-source software written in Python. To communicate with each sensor, the pyserial library was used; hence, any device that has a serial port can be added to the modular system so long as the software is modified to accommodate the device. The act of collecting data from each sensor simultaneously presented a unique challenge in that a typical computer program is sequential in its execution, meaning that data collection from one sensor should not be able to occur until collection from another sensor is finished. Furthermore, the runtime complexity of the code block associated with each sensor meant that data collection from one sensor could potentially interfere with the collection from another, and vice-versa. To circumvent these issues, the code blocks for each sensor were run in separate threads using the Python threading library. This allowed each code block to be executed simultaneously in parallel (as opposed to sequentially in series), meaning data collection from one sensor was independent of data collection from all other sensors. After the data was extracted, the code block for each sensor transformed the data string by removing superfluous characters and converting the pertinent data into a float format. To upload the transformed data to the Azure cloud database the sys, platform, and pyodbc libraries were used to communicate with the edge device and establish a database connection using an SQL driver.

In the case that an edge device is employed with an OS (operating system) distinct from Linux (the OS used by Raspberry Pi), the software will still function as it has been designed to function on both macOS and Windows as well. Furthermore, if one is unable to establish a database connection (e.g., lack of internet access, no SQL driver installed, etc.), the software can still be deployed in an offline-manner as it has been designed with a backup system that writes the data to CSV (comma-separated values) files using the csv library.

In addition to allowing offline data collection, this feature also aids in the prevention of data loss: if connection to the database is lost abruptly during perfusion, the data will continue to be written to the CSV files. In a similar case, it is also possible that the sensors could become disconnected (e.g., sensor turns off, serial port becomes disconnected) from the edge device. Typically, such an occurrence would cause a halting of data collection for that respective sensor—resulting in a cumbersome and unnecessary restart process of all sensors. However, the software resolves this potential issue by continuing to upload NULL values to the database and producing a warning sound (via the NumPy and the simpleaudio libraries) until the sensor is reconnected, ensuring a smooth and continuous data collection process. If more than one sensor becomes physically disconnected, the sensors must be reconnected according to the order stated by the GUI (graphical user interface).

The GUI of the software is designed to be efficient and user-friendly, employing methods from the time, datetime, and tkinter libraries. On the initial screen there are three windows: one for selecting the feature to be run, one for entering the UNOS (United Network for Organ Sharing) ID, and one for data output from the feature chosen.

The “Donor information upload” feature uses the given UNOS ID and the os library to search the edge device for the corresponding donor medical history file. This medical history file has a universal format, which is generated by UNOS for each individual donated organ. If found, the software will scan and upload pertinent information into a table on the edge device screen. The information may then be verified and edited as necessary before being uploaded to the cloud database. To make such an interactive tool possible, the pandas and pandastable libraries were utilized.

The “Blood gas data upload” feature allows one to manually input data collected from perfusate analysis assays. For our perfusion experiment, CG8+ and Renal Function Panel cartridges were used with iStat and Piccolo devices, respectively; however, the software can easily be modified to accommodate other perfusate assays.

Regarding the “Sensor data collection” feature, the facility of use contrasts with the complexity of the backend of the software. Prior to using, each sensor must be on and properly calibrated; the sensor devices are then connected according to the order stated on the “Data Output” window. When all devices are connected, one will be able to start and stop the data collection using the GUI. Once stopped, the data collection cannot be resumed; if one desires to restart the data collection or to choose another feature, the user can click the “Restart” icon in the bottom left of the screen.

The timestamps printed in the “Sensor Data Feed” window indicate the last time data was uploaded; this can allow one to diagnose issues that may arise during perfusion. If the time stamps cease to update, this indicates a loss of database connection (as mentioned prior) and a failure of the backup system, as the system is not infallible. In this case, the software can be exited and restarted. If the time stamps update irregularly, this is an indication that the sensors are out of order and must be reconnected in the proper order.

ii. Scalable Schema for Relational Data Storage and Analysis Adapted/Modified for Human Organ Research

As discussed above, the star schema model for ex vivo human organ research is a scalable and flexible solution for data storage and analysis. To implement this model, the relational database schema was made to be centered around a data table for the organs. This central data table includes the ID associated with each organ included in our studies. Furthermore, this table has a parent table, including all donor demographic information associated with that organ. This parent table is populated using the “Donor information upload” software feature implemented in the edge device, as described above.

For each device monitored by the edge device, a separate dimension table is created to store all metrics recorded. For instance, the pump speed, flow, and pressure were monitored by reading the serial output from the Medtronic Bio-console device. There is a dimension table corresponding to the Bio-console device, receiving information every time pump speed, flow, and pressure information from this device is captured.

An Azure SQL server was utilized to create this relational database. An Azure Subscription created and monitored by Yale Information Technology services was utilized, to ensure security and adherence to institutional guidelines for data storage and communication. The edge devices uses Open Database Connectivity (ODBC) protocol to communicate with the SQL server and upload data in real-time. Specifically, the pyodbc Python package with appropriate drivers depending on the operating system was used to establish this connection.

iii. Interactive Data Visualization and Analysis

An interactive dashboard of the cloud data was developed using interactive data visualization software developed by Microsoft-Power BI. This software has the functionality to query data directly from the Azure SQL database using the credentials provided. A professional subscription to Power BI is required to share this dashboard with other users and have the ability to interact with the dashboard on PC or handheld devices.

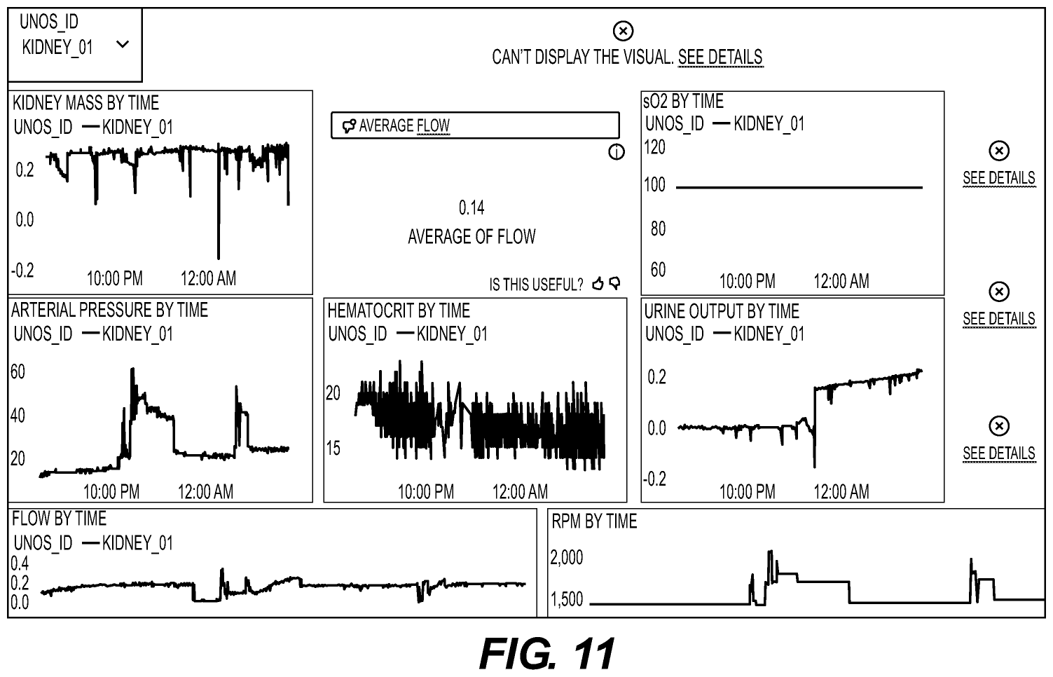

The developed dashboard displays kidney mass, arterial pressure, flow, hematocrit, pump RPM, oxygen saturation, and urine output in real-time, FIG. 11. Furthermore, demographic information such as age, weight, cold time, gender, and blood type are displayed using the information pulled from UNOS database, as outlined above.

As more kidney perfusion experiments are conducted, or if two kidneys from one donor are perfused at once, the dashboard is able to compare the perfusion metrics against previous or current ones. The plot legends are the UNOS ID, with each line in the plot representing a unique kidney. Therefore, if a dual kidney experiment is in progress and a treatment is added to one kidney, it is possible to compare the status of each kidney simultaneously.

Lastly, this dashboard has the functionality to type questions about the data using an artificial intelligence powered widget. For example, the user may ask simple questions such as: what is the average kidney mass. Furthermore, the user can ask more complex questions: how does the flow from the current kidney compare to average flow values for other kidney perfusion experiments.

### Results

i. Continuous Monitoring System

Flow, pressure, pump speed, kidney mass, urine mass, hematocrit, arterial oxygen saturation, Piccolo, and iStat measurements were uploaded to server continuously over the course of a 6-hour perfusion. The interactive dashboard was used to display data in realtime. There were no interruptions in the upload of data from the various sensors to the cloud database and displaying the results.

The kidney mass measurements displayed a significant amount of noise due to the measurement device being fixed on the same pole as the infusion devices. With every manipulation of infusion devices, the kidney moved slightly, causing noise/error in the measurement. In the future, the kidney would need to be fixed on a separate pole to fix this issue.

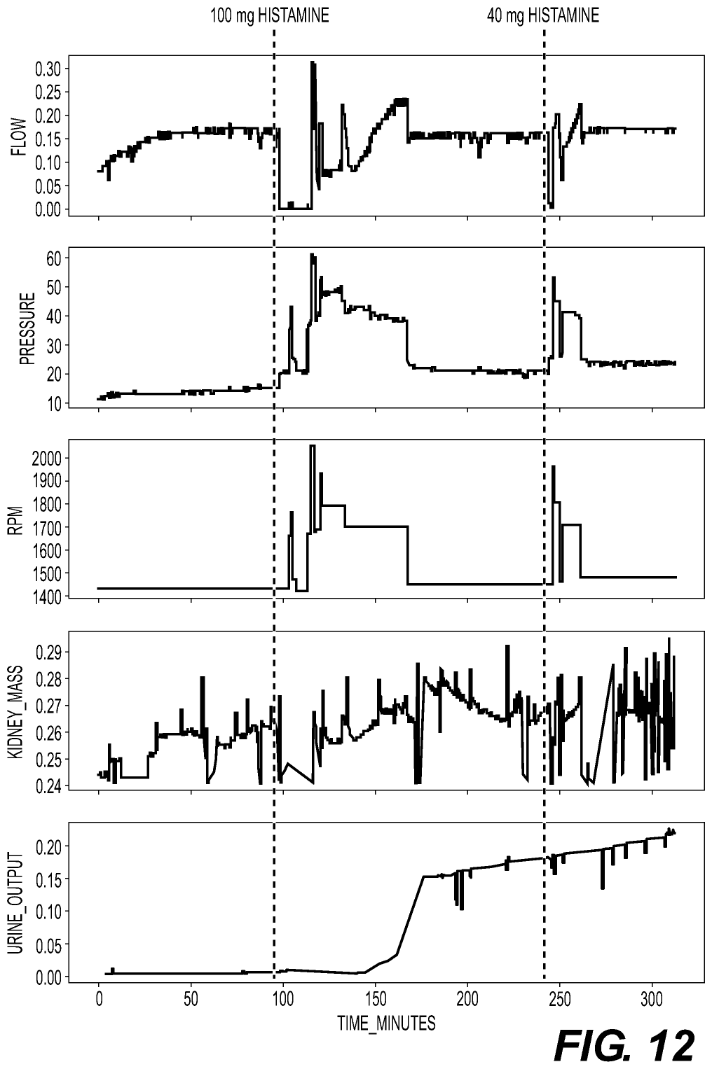

The earlier time points on the urine output plot (FIG. 12) do not reflect the correct urine output volume. During this time period, the urine would accumulate in the connecting tubes before falling into the urine bag and being registered by the weight measurement device. This issue can be addressed in the future by repositioning the urine bag and connecting tubes to prevent accumulation in the tubes.

ii. Histamine Administration

Histamine was administered at two different time-points during the perfusion. 100 mg of histamine administered on the arterial side at 90 minutes, which led to immediate halt of flow. Subsequently, the pump RPM was increased to 1750 but flow remained at zero. Then 20k units of heparin were added at 120 minute and slowly increased pump RPM to 2000. Flow slowly returned while arterial pressure was between 40-60 mmHg (compared to 15 mmHg starting arterial pressure). Flow slowly increased and arterial pressure normalized within the next 60 minutes (FIG. 12).

To see whether the same observation can be repeated, 40 mg of histamine was administered on the arterial side at 240 minutes. Flow immediately stopped afterwards. The pump RPM was slowly increased to 1900, without adding any heparin to the system, and flow slowly returned. Flow normalized over the next 30 minutes as displayed in FIG. 12.

Based on the manual recordings, the kidney weight quickly decreased after administration of histamine at 90 minutes—the weight dropped from approximately 0.26 Kg to approximately 0.24 Kg. As the flow and pressures normalized, the weight slowly became larger than before (0.27-0.28 Kg). The second administration of histamine lead to a similar weight change pattern.

Urine production was qualitative observed to initiate after the first histamine administration at 90 minutes. Histamine was initially chosen to potentially increase flow and induce edema in the organ, as suggested by previous studies (K. Ashina, Y. Tsubosaka, T. Nakamura, K. Omori, K. Kobayashi, M. Hori, H. Ozaki, and T. Murata, “Histamine Induces Vascular Hyperpermeability by Increasing Blood Flow and Endothelial Barrier Disruption In Vivo,” PLOS ONE, vol. 10, p. e0132367, July 2015). The aim was to capture the hypothesized increase in flow and test the utility of the compartmentalization mathematical model, described in the subsequent section, to estimate leak and edema. However, the observation was contradictory to this hypothesis. After arterial administration of histamine, the arterial pressure immediately increased leading to halt of flow. Furthermore, the kidney weight started to decrease.

Upon further investigation of the literature, it was evident that the immediate effect of histamine on the perfusion dynamics is complex and not yet fully understood (C. Grange, M. Gurrieri, R. Verta, R. Fantozzi, A. Pini, and A. C. Rosa, “Histamine in the kidneys: what is its role in renal pathophysiology?,” British Journal of Pharmacology, vol. 177, pp. 503-515, February 2020). For instance, four types of histamine receptors are reported to be present at various parts of the kidney and the activation of each receptor can lead to a variety of responses by the organ (C. Grange, M. Gurrieri, R. Verta, R. Fantozzi, A. Pini, and A. C. Rosa, “Histamine in the kidneys: what is its role in renal pathophysiology?,” British Journal of Pharmacology, vol. 177, pp. 503-515, February 2020).

For instance, histamine can cause vasoconstriction of the efferent arterioles, which are the small blood vessels that carry blood away from the glomeruli (C. Grange, M. Gurrieri, R. Verta, R. Fantozzi, A. Pini, and A. C. Rosa, “Histamine in the kidneys: what is its role in renal pathophysiology?,” British Journal of Pharmacology, vol. 177, pp. 503-515, February 2020). This vasoconstriction is believed to increase the pressure within the glomeruli and increase the GFR (C. Grange, M. Gurrieri, R. Verta, R. Fantozzi, A. Pini, and A. C. Rosa, “Histamine in the kidneys: what is its role in renal pathophysiology?,” British Journal of Pharmacology, vol. 177, pp. 503-515, February 2020). However, a discrepancy between GFR and urine output could be attributed to failure of tubular resorption as well as glomerular bypass.

In this case, pronounced vasoconstriction of the efferent arterioles may have occurred, thereby completely stopping the perfusate flow through the organ. After nearly doubling the pump RPM, the flow slowly returned. However, the arterial pressure, and thereby resistance, experienced by the kidney remained significantly elevated. Future system feedback to adjust RPM to stabilize pressure and/or flow automatically may be incorporated.

In conclusion, creating a cost-effective, modular, and open-source system for continuously monitoring the hemodynamics and function of organs outside of the body can yield crucial insights into their pathophysiology. With its open-source design, the system can be easily modified and expanded to accommodate additional sensors and assays in the future. By integrating real-time mathematical modeling with this system, microvascular leak, glomerular pressure, and other parameters that may have a direct correlation with clinical and pathological outcomes in the future can be estimated. Furthermore, these insights can pave the way for development of novel therapeutic strategies in the future.

Those skilled in the art will recognize, or be able to ascertain using no more than routine experimentation, many equivalents to the specific embodiments of the invention described herein. Such equivalents are intended to be encompassed by the following claims.

1.-77. (canceled)

78. A platform for collecting biological data from an ex vivo or in vivo normothermic perfused isolated organ, the platform comprising:
an organ perfusion machine; and
an edge device communicatively coupled to the organ perfusion machine, the edge device capable of capturing biological data from the perfusion machine during perfusion of the ex vivo or in vivo (e.g. in situ) normothermic perfused isolated organ, optionally wherein the platform collects and stores the biological data in an organ digital twin of the perfused isolated organ.

79. The platform of claim 78, communicatively coupled to a digital organ database optionally through the edge device and configured to transmit the biological data to the digital organ database.

80. A method comprising:
(a) using the platform of claim 78 for collecting one or more classes of biological data from an ex vivo or in vivo normothermic perfused instance of an isolated organ of interest, and
(b) storing the collected biological data in an organ digital twin of the organ of interest.

81. The method of claim 80:
(i) further comprising storing donor data in the organ digital twin, wherein the donor data is data related to the donor of the instance of the organ of interest, and/or
(ii) wherein the classes of biological data include one or more of physiologic data, genomic data, transcriptomic data, metabolomic data, proteomic data, lipidomic data, biopsy data, histological data, physical condition data, organ perfusion data, organ management data, and organ treatment data.

82. The method of claim 80, wherein:
(i) the genomic data includes one or more of whole exome sequencing and whole genome sequencing, and/or
(ii) the transcriptomic data includes one or more of bulk RNA sequencing, single cell RNA sequencing, single nuclear RNA sequencing, spatial RNA sequencing, and fluorescent in situ hybridization, and/or
(iii) the metabolomic data includes one or more of unbiased metabolomics, targeted analysis, metabolite profiling, metabolic fingerprinting, and spatial metabolomic imaging, and/or
(iv) the proteomic data includes one or more of targeted protein microarrays, ELISA, unbiased proteomics, and Luminex assays, and/or
(v) the lipidomic data includes one or more of direct infusion mass spectrometry (MS) analysis, liquid-phase separations coupled to MS, and desorption ionization techniques MS approaches (often used for mass spectrometry imaging (MSI), and/or
(vi) the histological data includes data generated using one or more of formalin fixed samples, paraffin embedded samples, fresh tissue section, frozen tissue section, histologic staining, histological imaging, standard histochemical stain, immunohistochemical stain, immunofluorescent stain, confocal microscopy, two-photon microscopy, epifluorescence, and light sheet microscopy, and/or
(vii) the physical condition data includes one or more of anatomic information related to the donor or anatomic information related to the instance of the organ, damage to the instance of the organ associated with recovery and preservation of the organ prior to initiation of perfusion, damage to the instance of the organ during perfusion, and damage to the instance of the organ following perfusion, and/or
(viii) the organ management data includes one or more of volume addition, mix of blood cells, crystalloid, and colloid in volume addition, volume removal, dialysis flow rate in, dialysis flow rate out, composition of dialysate, surgical intervention, nutritional maintenance, and additional maintenance infusion, and/or
(ix) the organ treatment data includes one or more of interventions to be evaluated, and/or
(x) the donor data includes one of more of blood gas analysis prior to organ recovery, labs prior to organ recovery, and summary data of the donor demographics and history, and/or
(xi) the point of care data includes one of more of blood gas analysis, metabolic panel analysis, and GEM blood analyzer,
optionally wherein:
(a) the surgical intervention includes one or more of cautery and sutures,
(b) the nutritional maintenance includes one or more of type of nutrition and flow rate of infusion, or
(c) the additional maintenance infusion includes one or more of bile salts and heparin.

83. The method of claim 82, wherein the intervention:
(i) includes one or more perturbations of the system, and/or
(ii) is chosen to establish or distinguish a link between modes of failure and types of intervention.

84. The method of claim 82, wherein:
(i) the blood gas analysis is collected from one or more of iSTAT, CHEM8, and CG4+, and/or
(ii) the metabolic panel analysis is from PICOLLO system, and/or
(iii) the blood analysis includes one or more of freezing point osmometer and biomarker analysis.

85. The method of claim 80, wherein,
(i) prior to storing the data, the organ digital twin comprised donor data, point of care data, and/or one or more classes of biological data, collected from or obtained for one or more different instances of the organ of interest, wherein the organ digital twin comprising data of the instance of the organ of interest and data collected from or obtained for one or more different instances of the organ of interest constitutes a collective organ digital twin, or
(ii) the donor data, point of care data, and biological data stored in the organ digital twin is collected from or obtained for only the instance of the organ of interest, wherein the organ digital twin comprising data of the instance of the organ of interest constitutes an individual organ digital twin.

86. The method of claim 80:
(i) further comprising analyzing the data in the organ digital twin to determine the condition of the instance of the organ of interest, and/or
(ii) wherein an alert is generated if a condition of the instance of the organ of interest determined by the analysis of the organ digital twin indicates that the instance of the organ of interest needs mechanical or therapeutic intervention, and/or
(iii) further comprising altering the ex vivo or in vivo perfusion conditions for the instance of the isolated organ of interest based on the condition of the instance of the organ of interest determined by the analysis of the organ digital twin, and/or
(iv) further comprising performing a mechanical intervention on the instance of the organ of interest based on the condition of the instance of the organ of interest determined by the analysis of the organ digital twin, and/or
(v) further comprising treating the instance of the organ of interest based on the condition of the instance of the organ of interest determined by the analysis of the organ digital twin to rehabilitate the instance of the organ of interest, and/or
(vi) further comprising analyzing all or a portion of the data comprised in the organ digital twin to produce derivatized data from the organ digital twin.

87. The method of claim 80 further comprising analyzing the data in the organ digital twin to determine one or more modes of failure of the instance of the organ of interest, and optionally treating, ex vivo and/or in vivo, the instance of the organ of interest based on one or more of the determined modes of failure.

88. The method of claim 87, wherein:
(i) analyzing the data identifies a logical connection between one or more modes of organ failure to one or more forms of therapeutic intervention, and/or
(ii) the determined modes of failure are in one or more classes of modes of failure, optionally wherein the classes of modes of failure include one or more of perfusion device failure modes, vascular failure modes, metabolic failure modes, immunological failure modes, and surgical failure modes,
optionally wherein:
(a) classes of modes of failure include one or more of perfusion device failure modes, vascular failure modes, metabolic failure modes, immunological failure modes, and surgical failure modes,
(b) the perfusion device failure modes include one or more of hypotension, hemorrhage, low hematocrit, low pH, and high potassium,
(c) the vascular failure modes include one or more of venous hypertension, arterial hypertension, non-device-related hypotension, edema, and microvascular obstruction,
(d) the metabolic failure modes include one or more of lactic acidosis, metabolic alkalosis, respiratory acidosis, respiratory alkalosis, and succinate-mediated electron transport disruption,
(e) the immunological failure modes include one or more of dysfunctional regulated cell death, dysfunctional IL-1-mediated inflammation, dysfunctional THF-mediated inflammation, and excessive damage-associated molecular pattern release, or
(f) the excessive damage associated molecular pattern release involves release of one or more of HMGB1, cell free DNA, ATP, and uric acid.

89. The method of claim 80 further comprising treating the instance of the organ of interest with a proposed therapy, collecting additional biological data from the instance of the organ of interest, storing the collected additional biological data in the organ digital twin, and analyzing the data in the organ digital twin to determine one or more of the effects of the proposed therapy on the instance of the organ of interest, and optionally storing the determined effects in the organ digital twin.

90. The method of claim 89 further comprising treating the instance of the organ of interest with the therapy of interest, collecting additional biological data from the instance of the organ of interest, storing the collected additional biological data in the organ digital twin, and analyzing the data in the organ digital twin to determine one or more of the effects of the therapy of interest on the instance of the organ of interest, and optionally storing the determined effects of the therapy of interest in the organ digital twin.

91. The method of claim 80 further comprising:
(i) analyzing the data in the organ digital twin to predict one or more effects on the organ of interest of a therapy of interest, or
(ii) integrating transplant recipient data with all or a portion of the data comprised in the organ digital twin and/or all or a portion of data derived from the organ digital twin, optionally
(a) further comprising analyzing the integrated transplant recipient data and organ digital twin data to assess suitability of the instance of the organ of interest for transplant into the recipient and/or
(b) analyzing the integrated transplant recipient data and organ digital twin data to assess suitability of the instance of the organ of interest for transplant into the recipient.

92. The method of claim 91 further comprising treating the instance of the organ of interest with the therapy of interest, collecting additional biological data from the instance of the organ of interest, storing the collected additional biological data in the organ digital twin, and analyzing the data in the organ digital twin to determine one or more of the effects of the therapy of interest on the instance of the organ of interest, and optionally storing the determined effects of the therapy of interest in the organ digital twin.

93. The method of claim 80 further comprising transmitting and/or displaying, in real time and/or as a static record, all or a portion of the data comprised in the organ digital twin and/or all or a portion of data derived from the organ digital twin, optionally wherein the data derived from the organ digital twin comprises the condition of the instance of the organ of interest, a generated alert, altered ex vivo or in vivo perfusion conditions, a mechanical intervention, and/or actions or interventions suggested by the analysis of data in the organ digital twin.

94. An organ digital twin produced by the method of claim 80.

95. A method of modeling responses of an organ of interest, the method comprising analyzing the response of the organ digital twin of claim 94 to an action of interest, optionally wherein:
(i) the action of interest is a change in one or more of the data comprised in the organ digital twin and/or in one or more of the data derived from the organ digital twin, and/or
(ii) the organ digital twin is stored in a digital physical medium.

96. The method of claim 80, further comprising transmitting all or a portion of the data comprised in the organ digital twin and/or all or a portion of data derived from the organ digital twin, and using a machine learning module to compute the current health state for the instance of the organ of interest.

97. The method of claim 96, wherein:
(i) the machine learning module is configured to train a machine learned model based on the transmitted data, and/or
(ii) the transmitted data includes donor data, point of care data, and/or one or more classes of biological data, and/or
(iii) computing the current health state for the organ of interest comprises using enriched data, and/or
(iv) the machine learning module is configured to train a machine learned neural network model, a Bayesian model, an artificial intelligence system, a rules-based system, or a combination thereof, and/or
(v) the machine learning module comprises neural networks selected from recurrent neural networks, convolutional neural networks, and artificial neural networks, optionally wherein the enriched data is from the same type of organ from the same donor or different donors.
